(19) United States
(12) Patent Application Publication
(10) Pub. No.: US 2004/0258620 A1
(43) Pub. Date: Dec. 23, 2004

(54) (Ethylene)-( propylene)-triaminepentaacetic acid derivatives, process for their production, and their use for the production of pharmaceutical agents

(76) Inventors: Lutz Lehmann, Berlin (DE); Ulrich Niedballa, Berlin (DE); Hannelore Niedballa, Berlin (DE); Johannes Platzek, Berlin (DE); Matthias Friebe, Berlin (DE); Christoph Hilger, Berlin (DE); Andre E Merbach, Pully (CH); Eva Jakab Toth, Chavernnes-Renens (CH); Robert Ruloff, Chavennes-Renens (CH); Sabrina Laus, Lausanne (CH)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/481,272

(22) PCT Filed: Jun. 24, 2002
(86) PCT No.: PCT/EP02/06963

Related U.S. Application Data
(60) Provisional application No. 60/300,479, filed on Jun. 22, 2001.
(60) Provisional application No. 60/305,936, filed on Jul. 17, 2001.
(60) Provisional application No. 60/328,108, filed on Oct. 10, 2001.
(60) Provisional application No. 60/335,106, filed on Oct. 23, 2001.

(30) Foreign Application Priority Data
Jul. 10, 2001 (DE) 101 33 435.4
Jul. 18, 2001 (EP) 01117355.6

(51) Int. Cl. A61K 49/00; C07F 5/00; C07F 15/02

(52) U.S. Cl. 424/9.364; 534/16; 544/225; 544/399

(57) ABSTRACT

The invention relates to a novel class of ligands, complexes comprising such ligands and a metal ion, and adducts of these metal complexes and a macromolecule. Pharmaceutical compositions and methods of making and using the ligand-metal complexes are also described. The invention also relates to the use of macromolecular adducts for enhancement of diagnostic imaging. In particular, the invention relates to (ethylene)-(propylene)-triaminepentaacetic acid (EPTPA) derivatives, a process for their production, and their use for the production of pharmaceutical agents for NMR diagnosis or radiodiagnosis or radiotherapy.

The invention relates to a novel class of ligands, complexes comprising such ligands and a metal ion, and adducts of these metal complexes and a macromolecule. Pharmaceutical compositions and methods of making and using the ligand-metal complexes are also described. The invention also relates to the use of macromolecular adducts for enhancement of diagnostic imaging. In particular, the invention relates to the subjects that are characterized in the claims, i.e., (ethylene)-(propylene)-triaminepentaacetic acid (EPTPA) derivatives, process for their production, and their use for the production of pharmaceutical agents for NMR diagnosis or radiodiagnosis or radiotherapy.

X-rays have long been used to produce images of human and non-human animal tissue, e.g., the internal organs of a patient. Typically, the patient is positioned between a source of X-rays and a film sensitive to the rays. Where organs interfere with the passage of the rays, the film is less exposed and the resulting developed film is indicative of the state of the organ. More recently, nuclear magnetic resonance (NMR) has been applied in medical imaging as magnetic resonance imaging (MRI). MRI avoids the harmful effects sometimes associated with exposure to X-rays.

To improve the quality of such diagnostic images, patients are often given image enhancers, or contrast agents, prior to image acquisition. For example, in X-ray diagnostics, increased contrast of internal organs such as the kidneys, the urinary tract, the digestive tract and the vascular system of the heart may be obtained by administering a radiopaque agent to the patient. In conventional proton MRI diagnostics, increased contrast of internal organs and tissues may be obtained by administering compositions containing paramagnetic metal species, which increase the relaxation rate of surrounding protons. In ultrasound diagnostics, improved contrast is obtained by administering compositions having acoustic impedances which are different than that of blood or other tissues.

Many interesting contrast agents are those in which organic acid ligands are coordinated to a metal atom or cation. The nature of substituents of the ligand, or complexing agent, can have a significant impact on tissue specificity of the contrast agent. For example, hydrophilic complexes tend to concentrate in the interstitial fluids, whereas lipophilic complexes tend to associate with cells. Thus, differences in hydrophilicity can lead to different applications of the compounds. The metal-ligand complex may be charged or neutral, and the charge may be altered to affect solubility.

If not all of the hydrogen atoms of an acidic ligand are substituted by the central (metal) ion (or central ions), it may be advantageous to increase the solubility of the complex salt by substituting the remaining hydrogen atoms with cations of inorganic and/or organic bases or amino acids. For example, the hydroxides, carbonates or bicarbonates of sodium, potassium or lithium are suitable inorganic cations. Suitable cations of organic bases include, among others, those of primary, secondary or tertiary amines, for example, ethanolamine, diethanolamine, morpholine, glucamine, N,N-dimethylglucamine or especially N-methylglucamine. Lysines, arginines or ornithines are suitable cations of amino acids, as generally are those of other basic naturally occurring such acids. If the complex salts contain several free acid groups, it is then often advantageous to produce neutral mixed salts which contain both inorganic and organic cations as counterions.

The complexing agents can also be coupled as conjugates with biomolecules that are known to concentrate in a particular organ or the part of an organ to be examined. These biomolecules include, for example, hormones (e.g., insulin), prostaglandins, steroid hormones, amino sugars, peptides, proteins, lipids, etc. Conjugates with albumins (e.g., human serum albumin) or antibodies, (e.g., monoclonal antibodies specific for tumor associated antigens or proteins such as myosin, etc.) are especially notable. The diagnostic agents formed therefrom can be used, e.g., to diagnose tumors and mycoardial infarctions. Conjugates with liposomes, or inclusion of salts of the contrast agent in liposomes, (e.g. unilamellar or multilamellar phosphatidylcholine-cholesterol vesicles) are suitable for liver imaging.

For X-ray diagnosis, the central ion in the contrasting agent is derived from an element with a high atomic number in order to promote sufficient absorption of X-rays. Diagnostic media containing a physiologically well tolerated complex salt containing a central ion chosen from elements with atomic numbers of 57 to 83 are suitable for this purpose. These include, for example, lanthanum(III), and other di- and tri-valent ions of the lanthanide group, gold(III), lead(II) or, especially, bismuth(III).

Chemical compounds with paramagnetic central ions are useful for MRI imaging. Two important MRI imaging parameters are spin-lattice (T1) and spin-spin and spin-echo (T2) relaxation times. The relaxation phenomena are essentially mechanisms whereby the initially imparted radio-frequency energy is dissipated to the surrounding environment. Thus, relaxation times are influenced by the environment of the nuclei, (e.g., viscosity, and temperature). The rate of energy loss, or relaxation, can also be influenced by neighboring paramagnetic nuclei. As such, chemical compounds incorporating paramagnetic nuclei may substantially alter the T1 and T2 values for nearby protons.

Nuclei which are useful in MRI contrasting agents include organic free radicals or transition or lanthanide metals which have from one to seven unpaired electrons. In general, paramagnetic species such as ions of elements with atomic numbers of 21 to 29, 42 to 44 and 58 to 70 are effective. Examples of suitable ions include chromium(III), manganese(II), manganese(III), iron(II), iron(III), cobalt(II), nickel(II), copper(II), praseodymium(III), neodymium(III), samarium(III), and ytterbium(III). Because of their very strong magnetic moments, gadolinium(III), terbium(III), dysprosium(III), holmium(III), and erbium(III) are preferred. Gadolinium(III) ions have been particularly useful as MRI contrasting agents.

Typically, paramagnetic ions have been administered in the form of complexes with organic complexing agents. A necessary prerequisite of any ligand that binds a metal to form a contrast agent is that the resulting contrast agent be stable so as to prevent the loss of the metal and its subsequent accumulation in the body. Such complexes provide the paramagnetic ions in a soluble, non-toxic form, and facilitate their rapid clearance from the body following the imaging procedure.

An example of a contrasting agent recognized in the art is gadolinium(III) with diethylenetriamine-pentaacetic acid (“DTPA”). Paramagnetic ions, such as gadolinium(III), have been found to form strong complexes with DTPA, ethylenediamine-tetra acetic acid (“EDTA”), and with tetra aza-cyclododecane-N,N′,N″,N′″-tetra acetic acid (“DOTA”). In addition to their use as contrast agents, lanthanide(III) poly-aminocarboxylates are also widely used as luminescent probes in fluoroimmunoassays.

Because the relaxivity of sphere-shaped molecules increases approximately linearly with molecular weight, and the relaxation of trivalent gadolinium, Gd(III),—containing contrast agents is mainly limited by their fast rotational motion, the incorporation of Gd chelates in large structures slows their rotational motion and increases relaxivity properties. Such structures can include polymers (Desser, et al., J. Magn. Reson. Imaging 1994, 4, 467); dendrimers (Tacke, et al., J. Magn. Reson. Imaging, 1997, 7, 678); proteins (Lauffer and Brady, J. Magn. Reson. Imaging, 1985, 3, 11) and micelles (André, et al., Chem. Eur. J., 1999, 5,2977).

Several approaches for making contrasting agents utilize supramolecular chemistry (Aime, et al., Chem. Eur. J., 1999, 5, 1253; Jacques, et al., Coord. Chem. Rev., 1999, 185,451) and self-organization (André, et. al., Chem. Eur. J., 1999, 5, 2977). For example, poly-α-cyclodextrins have been used to bind gadolinium poly-aminocarboxylate chelates that contain a lipophilic phenyl tail (Aime, et al., Chem. Eur. J., 1999, 5,1253), and polymetallic gadolinium-containing structures have been self-assembled through secondary recognition involving octahedral transition metals (Jacques, et al., Coord. Chem. Rev., 1999, 185, 451).

One important approach to developing efficient MRI contrast agents can involve the association of monomers into reversible supramolecular structures. This is accomplished by exploiting short distance interactions, (i.e., hydrogen bonds, aromatic π-stacking and van der Waal's interactions), which can be used for molecular recognition based upon complementary size, shape and chemical functionalities. The monomers used for the formation of supramolecular contrast agents must be rigid enough to ensure good intermolecular contact between interacting surfaces and also must overcome the loss of translational entropy of the monomers upon aggregation.

Contrast agents can be plagued by the in vivo release of free metal ions from the complex, which can result in metal toxicity subject. The toxicity of paramagnetic metal complexes can be affected by the nature of the complexing ligands. Principal factors involved in the design of ligands for paramagnetic metal complexes include the thermodynamic stability constant of the metal-ligand complex (the affinity of the totally unprotonated ligand for the metal); the conditional stability constant (which is pH dependent and is important when considering stability under physiological pH); the selectivity of the ligand for the paramagnetic metal over other endogenous metal ions (e.g., zinc, iron, magnesium and calcium); and the structural features that make in vivo transmetallation reactions much slower than the clearance rate of the complex.

Nuclear magnetic resonance (NMR) is now an extensively used method of medical diagnosis that is exploited in in-vivo imaging with which bodily vessels and bodily tissue (including tumors) can be visualized via the measurement of the magnetic properties of the protons in the bodily water. To this end, e.g., contrast media are used that produce a contrast enhancement in the resulting images by influencing certain NMR parameters of the body protons (e.g., relaxation times T¹ and T²) or make these images readable only. Paramagnetic ions, such as, e.g., gadolinium-containing complexes (e.g., Magnevist®), are primarily used because of the effect of the paramagnetic ions on the shortening of the relaxation times.

The use of radiopharmaceutical agents for diagnostic and therapeutic purposes has also been known for a long time in the area of biological and medical research. In particular, radiopharmaceutical agents are used to visualize certain structures, such as, for example, the skeleton, organs or tissue. The diagnostic application requires the use of such radioactive agents, which are concentrated after administration specifically in the structures in patients that are to be studied. These radioactive agents that accumulate locally can then be traced, plotted or scintigraphed by means of suitable detectors, such as, for example, scintillation cameras or other suitable imaging processes. The dispersion and relative intensity of the detected radioactive agent marks the site of a structure in which the radioactive agent is found, and the presence of anomalies in structures and functions, pathological changes, etc., can be visualized.

In a similar way, radiopharmaceutical agents can be used as therapeutic agents to irradiate certain pathological tissues or areas. Such treatment requires the production of radioactive therapeutic agents, which accumulate in certain structures, organs or tissues.

Both paramagnetic ions, such as, e.g.: Gd³⁺, Mn²⁺, Cr³⁺, Fe³⁺, and Cu²⁺ and many metallic radionuclides cannot be administered as solutions in free form since they are highly toxic. To make these ions suitable for an in-vivo application, they are generally complexed. For example, in EP-A-0 071 564, i.a., the meglumine salt of the gadolinium(III) complex of the diethylenetriaminepentaacetic acid (DTPA) is described as a contrast medium for the NMR tomography. A preparation that contains this complex was approved worldwide as the first NMR contrast medium under the name Magnevist®. This contrast medium is dispersed extracellularly after intravenous administration and is excreted renally by glomerular secretion. A passage of intact cell membranes is virtually not observed. Magnevist® is especially well suited for the visualization of pathological areas (e.g., inflammations, tumors).

The known contrast media and radiotherapeutic agents cannot be used satisfactorily for all applications, however. Many of these agents thus are dispersed into the entire extracellular space of the body. To increase the efficiency of these agents in in-vivo diagnosis and therapy, an attempt is made to increase their specificity and selectivity, for example on target cells or desired areas and structures of the body. An improvement of these properties is to be achieved by, for example, coupling metal complexes to biomolecules according to the “Drug-Targeting” principle. Biomolecules that can be considered include antibodies, their fragments, hormones, growth factors and substrates of receptors and enzymes (DE 195 36 780 A1).

In recent years, the need for diagnostic agents- and therapeutic agents that accumulate specifically in diseased tissues has increased. In the coupling of complexing agents to selectively accumulating substances, it is often observed, however, that the complexing properties of the complexing agents deteriorate, so that a weakening of the complex stability can occur. In this respect, problems can arise if a physiologically relevant proportion of the toxic metal ions is released from the conjugate in vivo. In addition, a reduction of the specificity of the biomolecules by the conjugate formation in the chelating agents can result.

DTPA derivatives and their chelates with radioactive metal isotopes are disclosed in U.S. Pat. No. 5,248,764. The target specificity of these derivatives is achieved by coupling the DTPA via a carbonyl radical to a peptide. In this respect, this carbonyl radical for the complexing of the metal ion is lost, however, so that there is danger of an easier release of the toxic metal ion.

DTPA derivatives with a reactive side group, which is bonded to the methyl-carbon atom of a carboxymethyl side chain, are disclosed in EP-A-0 297 307. This has the advantage that none of the complex binding sites is blocked by the reactive side chain, with whose help the derivative can be coupled to, for example, a biomolecule. On the other hand, the reactive side chain in this position can exert an undesirable steric influence on the complexing and thus the complexing constants.

Other DTPA derivatives, which have, for example, a reactive benzyl group on an ethylene bridge and whose second ethylene bridge is also substituted, are disclosed in U.S. Pat. No. 4,831,175 and No. 5,124,471.

The chelating agent (ethylene)-(propylene)-triaminepentaacetic acid (EPTPA) was already described in DE 29 18 842 A1 for complexing heavy metal ions such as iron and manganese when bleaching wood pulp that can be used in the production of paper, where it is to facilitate the removal of such ions from the aqueous system that contains the wood pulp.

The use of the gadolinium(III) complex of EPTPA as an MRI contrast medium was described by Yun-Ming Wang et al. in J. Chem. Soc., Dalton Trans., 1998, 41134118. Moreover, this article discloses to one skilled in the art, surprisingly enough, that the gadolinium(III)-EPTPA complex has a stability constant that is comparable to the gadolinium(III)-DTPA complex. This was especially surprising, therefore, since because of the ethylene bridges in DTPA, in each case two adjacent nitrogen atoms form with the latter a sterically ideal 5-ring when complexing the, gadolinium ions, while a sterically less ideal 6-ring with the central gadolinium ion must be formed in EPTPA by the introduction of a propylene bridge.

In addition, it is desirable to make available agents for diagnosis and therapy that have as a high a target specificity as possible and that have as high an in-vivo stability as possible for the metal ions that are toxic in most cases.

An object of the invention was therefore to make available new agents for NMR diagnosis and radiodiagnosis as well as radiotherapy that do not exhibit the above-mentioned drawbacks and have in particular high in-vivo stability, good compatibility and primarily organ-specific properties. On the one hand, the retention in the organs that are to be examined is to be sufficient to obtain with a small dosage the number of images that are necessary for an unambiguous diagnosis, but, on the other hand, an excretion of the metals from the body that is as quick as possible and that is to a large extent complete is then to be ensured. The NMR contrast media also are to show high proton relaxivity and thus allow a reduction of the dose in the case of an increase in signal intensity.

The invention provides a novel class of ligands, complexes comprising such ligands and a metal ion, and adducts in which these metal complexes are coupled (covalently or non-covalently) to a macromolecule. Pharmaceutical compositions and methods of making and using the ligand-metal complex and the macromolecular adducts for enhancement of diagnostic imaging are also described.

These metal-ligand complexes and their macromolecular adducts are useful as MRI contrast agents, diagnostic agents in X-ray, ultrasound or scintigraphic image analysis, as radiotherapy agents, and as luminescent probes. Because the macromolecular adducts have an unexpectedly high relaxivity, much less of the complex is required to be administered to the subject relative to commonly used image enhancing agents.

The compounds of the invention are chelating ligands which provide optimized water exchange rates of their Gd(II) complexes.

For example, tetra aza-cyclododecane-N,N′,N″,N′″-tetra acetic acid (“DOTA”) is a contrast enhancing agent commonly used in the art of magnetic resonance imaging. This ligand is modified so that at least one of the carboxylate arms has been lengthened by one methylene (—CH₂—) unit, as shown by Formula IIIa or the backbone is widened by one methylene (—CH₂—) unit, as shown by Formula IIa.

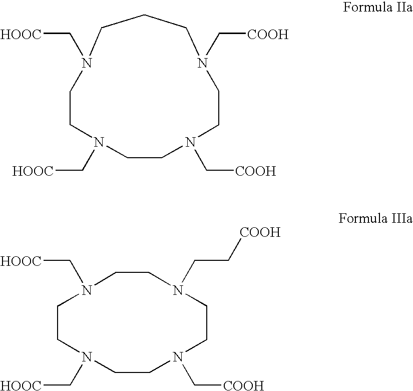

In another example of a contrasting agent recognized in the art is gadolinium(III) with diethylenetriamine-pentaacetic acid (“DTPA”). At least one of the carboxy arms can be modified, as demonstrated in Formulae IVa and Va.

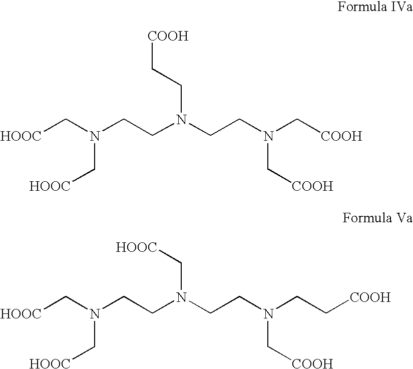

In some embodiments, at least one portion of the backbone can be modified instead of, or in addition to the carboxylate arm. An example is shown by Formula Ia.

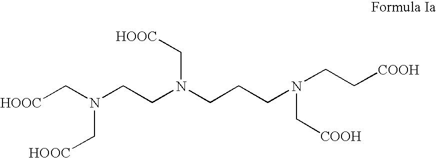

A further example is shown by EPTPA, Formula Ib.

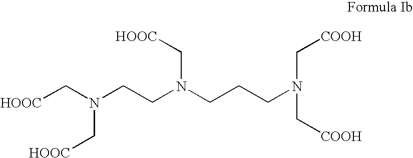

The above compounds can be modified so as to facilitate covalent or non covalent association with a macromolecule. The macromolecule can be any biologically compatible molecule such as proteins, carbohydrates, lipids, or any synthetic, biocompatible materials. The chelate linking groups are referred to herein as Z″. One example of a Z″ group is a benzyl isothiocyanate group.

In some examples, chelate ligands can be synthesized from molecules containing a nitro group, which can then be modified for use as a linker group, for example by conversion to an isothiocyanate group, which can function as a linker to couple the ligand to macromolecules according to well-documented procedures.

For example, the ligand EPTPA-bz-NO₂ (Formula 1) has been synthesized as described in FIG. 1. The chelate contains a group which can function as a linker to couple the ligand to macromolecules according to well-documented procedures. One example of such a group is an isothiocyanate group. The macromolecules can be biological molecules such as proteins, carbohydrates, lipids, or any synthetic, biocompatible materials.

The invention also provides a method of magnetic resonance imaging by administering to a human or non-human animal subject a contrast medium that includes a physiologically compatible paramagnetic metal complex of the herein described ligands and a non-toxic, pharmaceutically acceptable carrier, adjuvant or vehicle in an amount sufficient to allow for the generation of a magnetic resonance image of at least a part of the subject.

Further according to the invention, a method of diagnostic imaging is provided which comprises administering to a human or non-human animal subject a diagnostic agent comprising a physiologically compatible heavy metal complex of the present invention and a non-toxic, pharmaceutically acceptable carrier, adjuvant or vehicle, and generating an X-ray, ultrasound or scintigraphic image of at least a part of the subject.

Further according to the invention, a method of radiotherapy practiced on a human or non-human animal subject is provided which comprises administering to the subject a radioactive agent comprising a physiologically compatible radioactive metal complex of the present invention and a non-toxic, pharmaceutically acceptable carrier, adjuvant or vehicle.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 shows a scheme for the synthesis of one example of the class of ligands of the invention.

FIG. 2 shows the NMRD profiles of Gd(EPTPA-bz-NO₂) at 37° C. (bottom curve) and 25° C. (top curve).

FIG. 3 shows the ¹⁷O NMR, NMRD and EPR experimental data (points) and the fitted curves (lines) for Gd(EPTPA-bz-NO₂). 3A: reduced transverse (i=2) and longitudinal (i=1) ¹⁷O relaxation rates (B=9.4 T), 3B: reduced ¹⁷O chemical shifts (B=9.4 T). 3C: NMRD profiles. 3D: transverse electron spin relaxation rates at 0.34 T, as measured by EPR.

FIG. 4 shows the reduced transverse and longitudinal ¹⁷O relaxation rates and chemical shifts measured on Gd(TRITA-bz-NO₂). The solid lines represent the fitted curves. B=9.4 Tesla

FIGS. 5 and 6 show the titration curves of DPTPA, EPTPA-Bz-NO₂ and of their complexes.

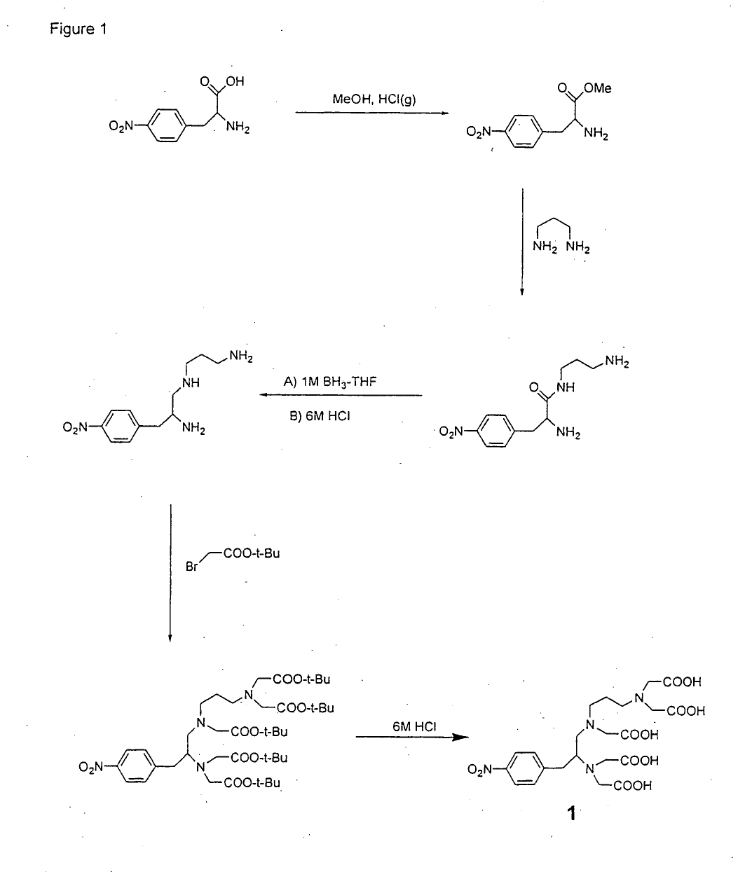

It has now been found that the above problems can be solved by compounds of formula V

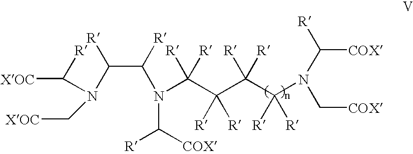

wherein n is 0 or 1; R′ are independently selected from the group consisting of a) functionalities suitable for coupling with a biocompatible macromolecule or biomolecule or b) non-coordinating substituents such as R²—R⁹ as defined below and at least one of R′ is a functionality suitable for coupling with a biocompatible macromolecule or biomolecule, whereby two of the R′ in the propylene or butylene unit can be part of a 5- or 6-membered ring; and X′ are independently selected from the group consisting of OZ (wherein Z stands for a hydrogen atom or a metal ion equivalent) or NR₂ (wherein each R is a non-coordinating substituent); with the provisio that at least two of X′ are OZ;

or a salt, hydrate, ester, solvate, prodrug, metabolite, stereoisomer, or mixture thereof.

The present invention also provides a novel class of ligands falling within Formulae IIIa, IVa and Va, as set forth above, wherein at least one carboxylate linker arm has been modified to increase the arm length by at least one methylene unit.

The invention also includes ligands of Formulae Ia, Ib and IIa, as set forth above, wherein at least one ethylene unit in the backbone has been lengthened by at least one methylene unit.

The invention also includes ligands of Formulae IIa and IIIa, as set forth above, which are modified to include non-coordinating substituents such as R′ as defined above.

The invention also includes the above-described ligands modified to include a functional group suitable for coupling the ligand to a macromolecule. Preferred macromolecules are biologically compatible macromolecules. In some aspects, the compounds of the invention fall with in Formula VIII:

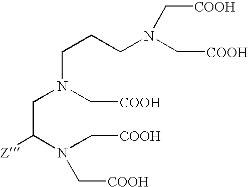

wherein:

Z′″ is, a benzyl group, a nitrobenzyl group or a functionality (e.g. an isothiocyanate group) suitable for coupling with a biological material or any biocompatible macromolecule.

In particular, the above problem is solved by conjugates that consist of biomolecules with (ethylene)-(propylene/butylene)-triaminepentaacetic acid derivatives, whose ethylene bridge is substituted with a reactive benzyl group, and whose propylene bridge or butylene bridge has additional substituents. The invention thus relates to compounds of general formula I

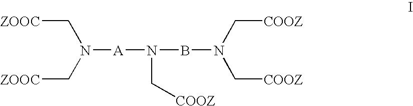

in which

Z stands for a hydrogen atom or a metal ion equivalent,

A stands for a radical of formula

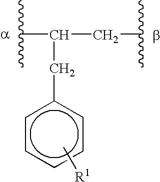

in which positions α and β that are characterized by

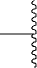

are bonded to any of the adjacent nitrogen atoms, R¹ is a nitro group or a group that can enter into a reaction with a biomolecule, and

B stands for a radical of formula

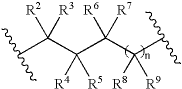

in which n is 0 or 1, and R², R³, R⁴, R⁵, R⁶, R⁷, R¹ and R⁹, independently of one another, are selected from a hydrogen atom, a straight-chain or branched, saturated or unsaturated C₁₋₆ alkyl group, which optionally can be substituted with 1 or 2 hydroxy groups and/or can contain 1 or 2 oxygen atoms, and an aralkyl group, whose aryl radical optionally can be substituted with an alkyl or alkoxy group, whereby two of radicals R², R³, R⁴, R⁵, R⁵, R⁷, R⁸ and R⁹ can be part of a 5- or 6-membered ring, provided that at least one and at most four of radicals R², R³, R¹, R⁵, R⁶, R⁷, Re, and R⁹ are not hydrogen atoms, as well as salts thereof, preferably with inorganic or organic bases.

Because of the reactive benzyl radical, the compounds according to the invention are suitable for the formation of conjugates with biomolecules, so that organ-specific properties are easily imparted to them, and the latter can be varied simply. Despite the substituted propylene or butylene bridge, metal complexes with the compounds according to the invention have high in-vivo stability. Moreover, the compounds according to the invention and their conjugates with biomolecules have good compatibility and good water solubility, so that they are suitable as pharmaceutical agents, especially for NMR diagnosis and radiodiagnosis as well as radiotherapy. The relaxivity of the complexes according to the invention is surprisingly high, so that the complexes, if they contain a paramagnetic ion, are especially well suited for NMR diagnosis.

In the compounds of Formula I according to the invention, A stands for a radical of formula

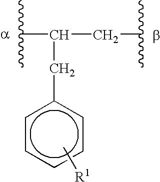

This radical can be bonded in positions α and β that are characterized by

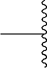

to any of the adjacent nitrogen atoms, i.e., the benzyl substituent of this radical can be adjacent to one of the two nitrogen atoms to which radical A is bonded. Radical A, however, is preferably bonded via position a to the (ZOOC—CH₂)₂—N radical, such that the benzyl substituent is adjacent to this radical.

The phenylene group of the benzyl substituent of radical A is substituted with a nitro group or a group that can enter into a reaction with a biomolecule. This substituent R′ is preferably in meta- or para-position, in particular bonded to the phenylene groups in para-position. The compounds of formula 1, in which R¹ is a nitrogen group, are especially well suited as intermediate compounds for the production of the compounds of formula 1, in which R¹ is a group that can enter into a reaction with a biomolecule.

Suitable groups that can enter into a reaction with a biomolecule are, for example, amino (—NH₂), isocyanate (—NCO), isothiocyanate (—NCS), hydrazine (—NHNH₂), semicarbazide (—NHCONHNH₂), thiosemicarbazide (—NHCSNHNH₂), chloroacetamide (—NHCOCH₂Cl), bromoacetamide (—NHCOCH₂Br), iodoacetamide (—NHCOCH₂₁), acylamino, such as, for example, acetylamino (—NHCOCH₃), maleimide, maleimidacylamino, such as, for example, 3-(2,5-dioxo-2,5-dihydro-pyrrol-1-yl)-propionylamino, activated esters, such as, for example,

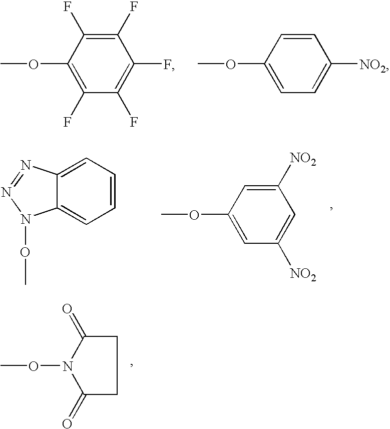

mixed anhydrides, azide, hydroxide, sulfonyl chloride and carbodiimide.

In formula 1, B stands for a radical of formula

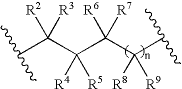

Hereinafter, n can be either 0 or 1, whereby compounds in which n=0 are preferred. The substituents R², R³, R⁴, R⁵, R⁶, R⁷, R¹ and R⁹ are, independently of one another, selected from a hydrogen atom, a straight-chain or branched, saturated or unsaturated C₁₋₅ alkyl group, which optionally can be substituted with 1 or 2 hydroxy groups and/or can contain 1 or 2 oxygen atoms, and an aralkyl group, whose aryl radical optionally can be substituted with an alkyl or alkoxy group. In this connection, at least one and at most four of these radicals must not be hydrogen atoms, so that the propylene bridge or butylene bridge B in the compounds of formula I according to the invention carries at least one and at most four substituents.

The substituent or the substituents of bridge B can be a C₁₋₆ alkyl group, which optionally can be substituted with 1 or 2 hydroxy groups and/or can contain 1 or 2 oxygen atoms. Preferably the C₁₋₆ alkyl group is a methyl, ethyl, n-propyl, iso-propyl, n-butyl, iso-butyl or tert-butyl group. At the same time or alternately, one or more substituents of bridge B can be an aralkyl group, whereby aryl-C₁₋₆ alkyl groups and especially benzyl are preferred. The aryl radical of these aralkyl groups can be substituted preferably in para-position with an alkyl or alkoxy group. Preferably this alkyl group is a C₁₋₆ alkyl group, such as especially methyl or ethyl, and this alkoxy group is a C₁₋₆ alkoxy group, such as especially methoxy or ethoxy.

Bridge B is preferably substituted with 1 or 2 methyl, ethyl or benzyl groups.

Also preferred are those compounds according to the invention in which R², R³, R⁴, R⁵, R⁶, R⁷, R⁸ and R⁹ are selected, so that B is symmetrical.

In addition or as an alternative, two of radicals R², R³, R⁴, R⁵, R⁶, R⁷, R⁸ and R⁹ can be part of a 5- or 6-membered ring. The number of ring members of such a ring include the carbon atoms, to which the ring-forming radicals R², R³, R⁴, R⁵, R⁶, R⁷, R⁸ and R⁹ are bonded, as well as carbon atoms of propylene bridge or butylene bridge B that are optionally found inbetween. The ring can be saturated or unsaturated; 5- and 6-membered saturated rings are preferred.

Preferred propylene or butylene bridges B are: —CH₂—CH₂—CH(CH₂—CH₃)—, —CH(CH₂—CH₃)—CH₂—CH₂—, —CH₂—C(CH₃)₂—CH₂—, —CH₂—C H(CH₃)—CH₂, —CH₂—CH(CH₂-phenyl)-CH₂—, —CH₂—CH(CH₃)—CH(CH₃)—CH₂—.

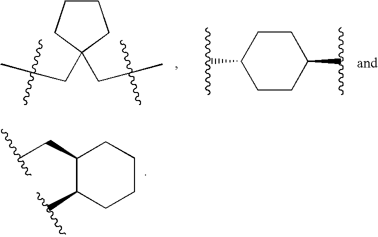

The compounds according to the invention contain at least one chirality center. Even if no distinction is made between the diferent enantiomers in the description and the claims, the above-mentioned compounds, if not otherwise indicated, always encompass both enantiomers and, in the presence of several stereo centers, also all possible diastereomers as well as mixtures thereof.

The compounds of formula I according to the invention are suitable for the production of conjugates with biomolecules. These conjugates have general formula II

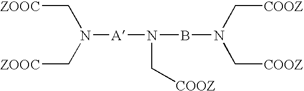

in which Z and B are defined above, and A′ stands for a radical of formula

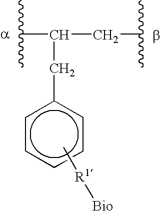

in which positions α and β that are characterized by

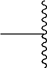

are bonded to any of the adjacent nitrogen atoms, and Bio stands for the radical of a biomolecule, which is bonded via radical R¹ of a reactive group to the phenylene ring, as well as salts thereof, preferably with inorganic or organic bases. Radical R^{1′} is preferably a radical R¹ as defined above after its reaction with a biomolecule.

“Biomolecule” is defined here as any molecule that either occurs naturally in, for example, the body or was produced synthetically with an analogous structure. Moreover, among the latter, those molecules are defined that can occur with a biological molecule that occurs, for example, in the body or a structure in interaction that occurs there, so that, for example, the conjugates can accumulate at certain desired spots of the body. “Body” is defined here as any plant or animal body, whereby animal and especially human bodies are preferred.

To form conjugates with the compounds according to the invention, the following biomolecules are especially suitable.

Biopolymers, proteins, such as proteins that have a biological function, HSA, BSA, etc., proteins and peptides that accumulate at certain spots in the organism (e.g., at receptors, cell membranes, ducts, etc.), peptides that can be cleaved by proteases, peptides with synthetic predetermined points of break (e.g., labile esters, amides, etc.), peptides that are cleaved by metalloproteases, peptides with photocleavable linkers, peptides with groups that can be cleaved with oxidative agents (oxydases), peptides with natural and unnatural amino acids, glycoproteins (glycopeptides), signal-proteins and antiviral proteins, synthetically modified biopolymers, such as biopolymers that are derivatized with linkers, modified metalloproteases and derivatized oxydase, etc., carbohydrates (mono- to polysaccharides), such as derivatized sugar, sugar that can be cleaved in the organism, cyclodextrins and derivatives thereof, amino sugar, chitosan, polysulfates and acetylneuraminic acid derivatives, antibodies, such as monoclonal antibodies, antibody fragments, polyclonal antibodies, miniborides, single chains (also those fragments that are linked by linkers to multiple fragments), red blood cells and other blood components, cancer markers (e.g., CAA) and cell-adhesion substances (e.g., Lewis X and Anti-Lewis X derivatives), DNA and RNA fragments, such as derivatized DNAs and RNAs (e.g., those that were found by the SELEX process, synthetic RNA and DNA (also with unnatural bases), PNAs (Hoechst) and antisense, β-amino acids (Seebach), vector amines for transfer into the cell, biogenic amines, pharmaceutical agents, oncological preparations, synthetic polymers, which are directed to a biological target (e.g., receptor), steroids (natural and modified), prostaglandins, taxol and derivatives thereof, endothelins, alkaloids, folic acid and derivatives thereof, bioactive lipids, fats, fatty acid esters, synthetically modified mono-, di- and tri-glycerides, liposomes that are derivatized on the surface, micelles that consist of natural fatty acids or perfluoroalkyl compounds, porphyrins, texaphrines, expanded porphyrins, cytochromes, inhibitors, neuramidases, neuropeptides, immunomodulators, such as FK 506, CAPE and gliotoxin, endoglycosidases, substrates that are activated by enzymes, such as calmodolin kinase, casein-kinase II, glutathione-S-transferase, heparinase, matrix-metalloproteases, β-insulin-receptor-kinase, UDP-galactose, 4-epimerase, fucosidases, G-proteins, galactosidases, glycosidases, glycosyltransferases and xylosidases, antibiotics, vitamins and vitamin analogs, hormones, DNA-intercalators, nucleosides, nucleotides, lectins, vitamin B12, Lewis-X and related substances, psoralens, dienetriene antibiotics, carbacyclins, VEGF (vascular endothelial growth factor), somatostatin and derivatives thereof, biotin derivatives, antihormones, dendrimers and cascade polymers as well as their derivatives, tumor-specific proteins and synthetic agents, polymers that accumulate in acidic or basic areas of the body (pH-controlled dispersion), myoglobins, apomyoglobins, etc., neurotransmitter peptides, tumor necrosis factors, peptides that accumulate in inflamed tissues, blood-pool reagents, anion and cation-transporter proteins, polyesters (e.g., lactic acid), polyamides and polyphosphates.

Most of the above-mentioned biomolecules are commercially available from, for example, Merck, Aldrich, Sigma, Calibochem and Bachem.

In addition, all “plasma protein binding groups” or “target binding groups” that are disclosed in WO 96/23526 and WO 01/08712 can be used as biomolecules. The content of these two laid-open specifications is therefore integrated by reference to this description.

The compounds according to the invention are also suitable for conjugation on all molecules that are reacted with fluorescence dyes in the prior art to determine, for example, their location by epifluorescence microscopy within the cell. After the administration of the medication, the compounds with, in principle, any medications can also be conjugated to then track the transport within the organism, for example by the NMR technique. It is also possible that the conjugates from the compounds according to the invention and the biomolecules contain other additional molecules, which had been conjugated on the biomolecules. The term “biomolecule” in terms of this invention thus encompasses all molecules that occur in the biological systems and all molecules that are biocompatible.

The compounds of general formula I and conjugates thereof with biomolecules can be obtained, for example, by reaction of a compound of formula III

H₂N-A-NH—B—NH₂ III

whereby A and B are as defined above, with a compound of formula IV

Nu-CH₂—COOZ′ IV,

whereby Nu stands for a nucleofuge and Z′ stands for a hydrogen atom, a metal ion equivalent, preferably an alkali or alkaline-earth metal, such as especially sodium or potassium, or a protective group for carboxyl. The compound that is thus obtained can then be reacted with a biomolecule, whereby radical R¹, if it is nitro, first must be converted into a group that can enter into a reaction with a biomolecule. After that, and after the removal of optionally still present protective groups, and in a way that is known in the art, a reaction can be performed, if desired, with at least one metal oxide or metal salt to obtain the desired metal complexes. In the complexes that are thus obtained, still present acidic hydrogen atoms, if desired, can then optionally be completely or partially substituted by cations of inorganic and/or organic bases, amino acids or amino acid amides.

As a nucleofuge, the radicals that are advantageously used are:

Cl, I, Br, —OTs, —OMs and —O-triflate.

The reaction is performed in a mixture of water and organic solvents, such as: isopropanol, ethanol, methanol, butanol, dioxane, tetrahydrofuran, dimethylformamide, dimethylacetamide, formamide or dichloromethane. Preferred are ternary mixtures that consist of water, isopropanol and dichloromethane.

The reaction is performed in a temperature range of between −10° C. and 100° C., preferably between 0° C. and 30° C.

The neutralization of optionally still present free carboxy groups is carried out with the aid of inorganic bases (e.g., hydroxides, carbonates or bicarbonates) of, e.g., sodium, potassium, lithium, magnesium, or calcium and/or organic bases, such as, i.a., primary, secondary and tertiary amines, such as e.g., ethanolamine, morpholine, glucamine, N-methylglucamine and N,N-dimethylglucamine, as well as basic amino acids, such as, e.g., lysine, arginine, and ornithine or amides of originally neutral or acidic amino acids.

For the production of neutral complex compounds, for example, enough of the desired bases can be added in acidic complex salts in aqueous solution or suspension to ensure that the neutral point is reached. The solution that is obtained can then be evaporated to the dry state in a vacuum. It is often advantageous to precipitate the formed neutral salts by adding water-miscible solvents, such as, e.g., lower alcohols (methanol, ethanol, isopropanol and others), low ketones (acetone and others), polar ethers (tetrahydrofuran, dioxane, 1,2-dimethoxyethane and others) and to obtain crystallizates that are easily isolated and readily purified. It has proven especially advantageous to add the desired base as early as during the complexing of the reaction mixture and thus to save a process step.

The production of the compounds of formula I according to the invention is explained below in the example of a preferred compound, in which R¹ is —NO₂, and B is R—CH₂—CH₂—CH(—CH₂—CH₃)—, and Z means hydrogen. The production of the compound

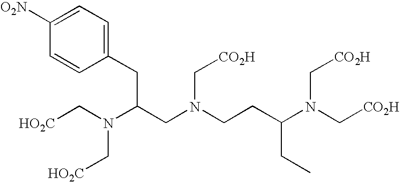

can be carried out from the t-butylesters 1

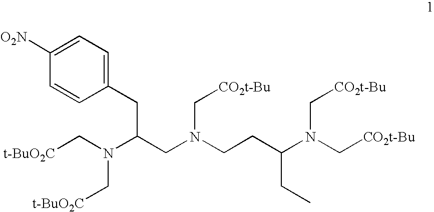

by acidic hydrolysis with trifluoroacetic acid. The compound of formula 1 can be obtained by alkylation of the amine of formula 2

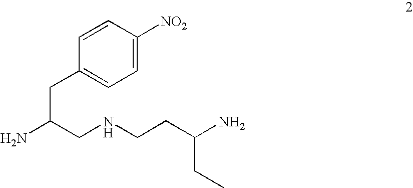

with bromoacetic acid-t-butyl ester. The ester can be obtained from Merck, Fluka or Aldrich.

Amine 2 can be obtained by hydrolysis with trifluoroacetic acid from desired compound 3

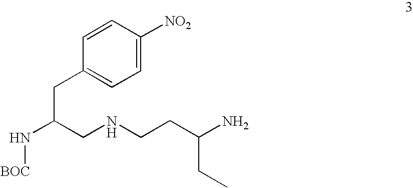

Compound 3 is available by alkylation of mesylate 4 with 1,3-diaminopentane 5 and chromatographic separation of the amino mixture.

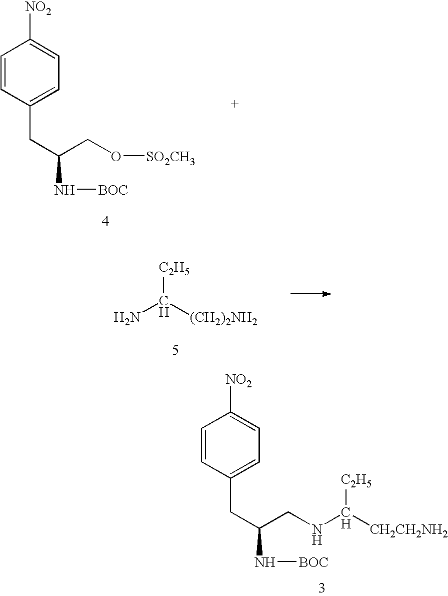

Amine 5 commercially available (Aldrich, Fluka).

Mesylate 4 can be obtained by reaction of alcohol 6 with methanesulfonyl chloride in the presence of triethylamine.

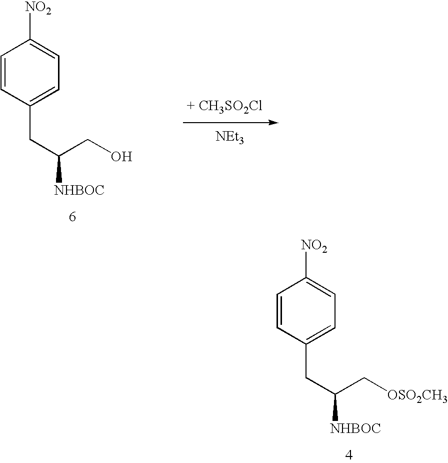

Alcohol 6 can be obtained by reduction of ester 7 with sodium borohydride in tetrahydrofuran/methanol (8:1):

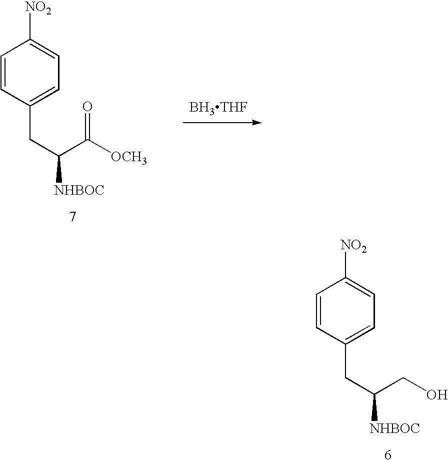

4-Nitrophenylalanine methyl ester 7 can be produced from corresponding acid 8 by esterification with methyl iodide in the presence of sodium bicarbonate in dimethylformamide:

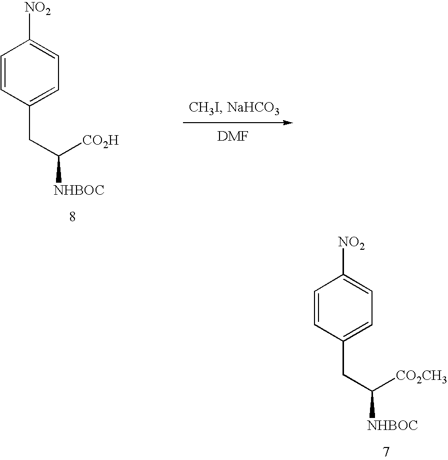

Acid 8 is commercially available (Aldrich, Fluka).

As an alternative, the procedure can also be such that component 3 is obtained by amide formation from phenylalanine 8 and 1-ethyl-1,3-propanediamine 5 and subsequent reduction of the amide bond. The chromatographic separation can be avoided, if the amine component is used as a 3-N-BOC derivative.

The α,ω-diamines that are required for the synthesis are available, for example, via the synthesis methods that are depicted grammatically below:

C-3 Diamines, Substituents at C-2

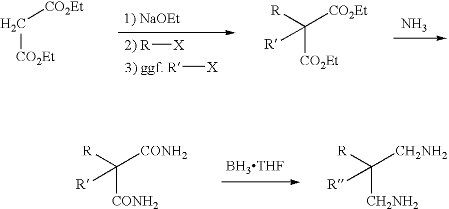

R: Alkyl, etc.

R¹: Hydrogen (then step 3 of the first stage is unnecessary) or R X: Halogen

C-3 Diamines, Substituent at C-1

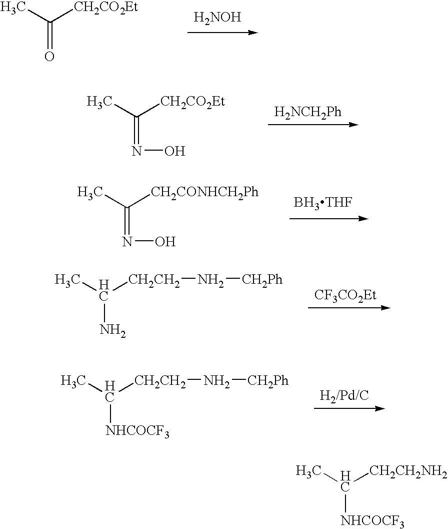

C4 Diamines. Substituent at C-4

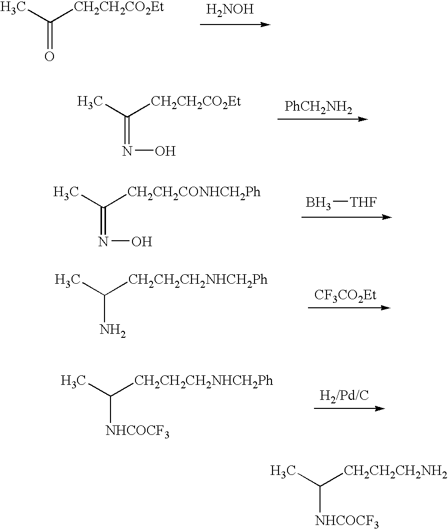

The conversion of the compounds of general formula I, in which R¹=NO₂, into compounds of general formula I, in which R¹ is not equal to nitro, is carried out according to known methods. The hydrogenation of the nitro group into the amino group is possible with, for example, 10% palladium on carbon (cf. EP 475 617; EP 173 629 and U.S. Pat. No. 5,087,696).

The amino group can be converted into the corresponding amide by reaction with nitrophenyl or hydroxysuccinimide esters. It can also be converted, however, by reaction with thiosphosgene in the isothiocyanate, which couples directly with amino groups to the thiourea. The isothiocyanate can also react with hydrazine to form thiosemicarbazide, which then is reacted specifically with the oxidized sugar molecules of an antibody to form thiosemicarbazide.

Analogously, amine is reacted with phosgene to form isocyanate, and the latter is reacted with hydrazine to form semicarbazone. The anilino group can also be acylated. If the reaction with the activated ester of the 4-maleimidobutyric acid (Fluka) is performed, a specific reagent that binds to —SH groups is obtained. The haloacetamides, which can be obtained by reaction of the anilines with haloactivated esters, also bind to —SH groups.

The amino group itself can also be used as a binding site for the carbonyl groups of oxidized sugar, if the partner molecule tolerates the conditions of reductive amination.

The production of complexes for the production of NMR diagnostic agents can be carried out in the way that was disclosed in Patents EP 71564, EP 130934 and DE-OS 34 01 052. To this end, the metal oxide or a metal salt (for example, chloride, nitrate, acetate, carbonate or sulfate) of the desired element in water and/or a lower alcohol (such as methanol, ethanol or isopropanol) is dissolved or suspended and reacted with the solution or suspension of the equivalent amount of the complexing agent according to the invention.

If the complexing agents are to be used for the production of radiodiagnostic or radiotherapeutic agents, the production of the complexes from the complexing agents can be carried out according to the methods described in “Radiotracers for Medical Applications,” Volume 1, CRC Press, Boca Raton, Fla.

It may be desirable to produce the complex only shortly before its use, especially if it is to be used as a radiopharmaceutical agent. The invention therefore also comprises a kit for the production of radiopharmaceutical agents encompassing a compound of formula I and a conjugate of formula II, in which Z is hydrogen, and a compound of a desired metal.

Subjects of the invention are also pharmaceutical agents that contain at least one physiologically compatible compound of general formula I or at least one physiologically compatible conjugate of general formula II, optionally with the additives that are commonly used in galenicals.

The production of the pharmaceutical agents according to the invention is carried out in a way that is known in the art, by the complex compounds—optionally with the addition of the additives that are commonly used in galenicals—being suspended or dissolved in aqueous medium, and then the suspension or solution optionally being sterilized. Suitable additives are, for example, physiologically harmless buffers (such as, e.g., tromoethamine), additions of complexing agents or weak complexes (such as, e.g., diethylenetriaminepentaacetic acid or the Ca complexes corresponding to the metal complexes according to the invention) or —if necessary—electrolytes, such as, e.g., sodium chloride or —if necessary—antioxidants, such as, e.g., ascorbic acid.

If suspensions or solutions of the agents according to the invention in water or physiological salt solution are desired for enteral administration or other purposes, they are mixed with one or more additive(s) that are commonly used in galenicals [e.g., methyl cellulose, lactose, mannitol] and/or surfactant(s) [e.g., lecithins, Tween®, Myrj®] and/or flavoring substance(s) for taste correction [e.g., ethereal oils].

In principle, it is also possible to produce the pharmaceutical agents according to the invention even without isolating the complex salts. In any case, special care must be taken to perform the chelation so that the salts and salt solutions according to the invention are virtually free of non-complexed metal ions that have a toxic effect.

This can be ensured, for example, with the aid of color indicators, such as xylenol orange, by control titrations during the production process. The invention therefore also relates to the process for the production of complex compounds and salts thereof. Purification of the isolated complex salt is a last safety measure.

The pharmaceutical agents according to the invention preferably contain 1 fmol-1.3 mol/l of the complex salt and are generally dosed in amounts of 0.0001-5 mmol/kg. They are intended for enteral and parenteral administration. The complex compounds according to the invention are used

1. For NMR diagnosis in the form of their complexes with the paramagnetic ions of the elements with atomic numbers 21-29, 42, 44 and 58-70. Suitable ions are, for example, the chromium(III), iron(II), cobalt(II), nickel(II), copper(II), praseodymium(III), neodymium(III), samarium(III) and ytterbium(III) ions. Because of their strong magnetic moment, the gadolinium(III), terbium(III), dysprosium(III), holmium(III), erbium(III), manganese(II) and iron(III) ions are especially preferred for NMR diagnosis.

2. For radiodiagnosis and radiotherapy in the form of their complexes with the radioisotopes of the elements with atomic numbers 26, 27, 29, 31, 32, 37-39, 43, 46, 47, 49, 61, 62, 64, 67, 70, 71, 75, 77, 82 and 83.

The agents according to the invention meet the many different requirements for suitability as contrast media for nuclear spin tomography. After oral or parenteral administration, they are thus extremely well suited for enhancing the information value of the image that is obtained with the aid of a nuclear spin tomograph by increasing the signal intensity. They also show the great effectiveness that is necessary to load the body with the minimum possible amounts of foreign substances, and the good compatibility that is necessary to maintain the non-invasive nature of the studies.

Good water-solubility and low osmolality of the agents according to the invention allow the production of highly concentrated solutions to keep the volume burden of the circulatory system within reasonable limits and to offset the dilution by bodily fluids, i.e., NMR diagnostic agents have to be 100 to 1000 times more water-soluble than for NMR spectroscopy. In addition, the agents according to the invention do not have only high stability in vitro, but also surprisingly high stability in vivo, such that a release or an exchange of the ions—that are inherently toxic—and that are not covalently bonded in the complexes takes place only extremely slowly within the time in which the new contrast media are completely excreted again.

In general, the agents according to the invention for use as NMR diagnostic agents are dosed in amounts of 0.0001-5 mmol/kg, preferably 0.005-0.5 mmol/kg. Details of use are discussed in, e.g., H.-J. Weinmann et al., Am. J. of Roentgenology 142, 619 (1984).

Low dosages (below 1 mg/kg of body weight) of organ-specific NMR diagnostic agents can be used, for example, for detecting tumors and myocardial infarction. Especially low dosages of the complexes according to the invention are suitable for use in radiotherapy and radiodiagnosis.

The complex compounds according to the invention can also be used advantageously as susceptibility reagents and as shift reagents for in-vivo NMR spectroscopy.

Because of their advantageous radioactive properties and the good stability of the complex compounds that are contained therein, the agents according to the invention are also suitable as radiodiagnostic agents and radiotherapeutic agents. Details of their use and dosage are described in, e.g., “Radiotracers for Medical Applications,” CRC Press, Boca Raton, Fla., 1983, as well as in Eur. J. Nucl. Med. 17 (1990), 346364 and Chem. Rev. 93 (1993) 1137-1156.

For SPECT, complexes with the isotopes ¹¹¹In and ^{99m}Tc are suitable.

Another imaging method with radioisotopes is the positron-emission-tomography, which uses positron-emitting isotopes, such as, e.g., ⁴³Sc, ⁴⁴SC, ⁵²Fe, ⁵⁵Co, ⁵⁸Ga, ⁶⁴Cu, ⁸⁶Y and ^{94m}Tc (Heiss, W. D.; Phelps, M. E.; Positron Emission Tomography of Brain, Springer Verlag Berlin, Heidelberg, N.Y. 1983).

The compounds according to the invention are also suitable, surprisingly enough, for differentiating malignant and benign tumors in areas without blood-brain barriers.

They are distinguished in that they are completely eliminated from the body and thus are well compatible.

Since the substances according to the invention accumulate in malignant tumors (no diffusion in healthy tissue, but high permeability of tumor vessels), they can also support the radiation therapy of malignant tumors. This is different from the corresponding diagnosis only by the amount and type of the isotope that is used. The purpose in this case is the destruction of tumor cells by high-energy short wave radiation with as small a range of action as possible. To this end, interactions of the metals that are contained in the complexes (such as, e.g., iron or gadolinium) are used with ionizing radiations (e.g., x rays) or with neutron rays. The local radiation dose at the site where the metal complex is found (e.g., in tumors) is significantly increased by these effects. To produce the same radiation dose in malignant tissue, the radiation exposure for healthy tissue can be considerably reduced when using such metal complexes and thus burdensome side effects for the patients can be avoided. The metal complex conjugates according to the invention are therefore also suitable as radiosensitizing substances in the radiation therapy of malignant tumors (e.g., use of the Mossbauer effects or in neutron capture therapy). Suitable β-emitting ions are, e.g., ⁴⁶Sc, ⁴⁷Sc, ⁴⁸Sc, ⁷²Ga, ⁷³Ga, ⁹⁰Y, ¹⁷⁷Cu, ¹⁰⁹Pd, ¹¹¹Ag, ¹⁴⁹Pm, ¹⁵³Sm, ¹⁶⁶Ho, ¹⁷⁷Lu, ¹⁸⁶Re and ¹⁸⁸Re, whereby ⁹⁰Y, ¹⁷⁷Lu, ⁷²Ga, ¹⁵³Sm and ⁶⁷Cu are preferred. Suitably short half-lives that have α-emitting ions are, e.g., ²¹¹At, ²¹¹Bi, ²¹²Bi, ²¹³Bi and ²¹⁴Bi, whereby ²¹²Bi is preferred. A suitable photon- and electron-emitting ion is ¹⁵⁸Gd, which can be obtained from ¹⁵⁷Gd by neutron capture.

If the agent according to the invention is intended for use in the variant of radiation therapy that is proposed by R. L. Mills et al. [Nature Vol. 336, (1988), p. 787], the central ion must be derived from a Möβbauer isotope, such as, for example, ⁵⁷Fe or ¹⁶¹Eu.

In the in-vivo administration of the therapeutic agents according to the invention, the latter can be administered together with a suitable vehicle, such as, e.g., serum or physiological common salt solution, and together with another protein, such as, e.g., human serum albumin. In this case, the dosage is dependent on the type of cellular disorder, the metal ion that is used and the type of imaging method.

The therapeutic agents according to the invention are administered parenterally, preferably i.v.

Details of the applications of radiotherapeutic agents are discussed in, e.g., R. W. Kozak et al., TIBTEC, October 1988, 262 (see above Bioconjugate Chem. 12 (2001) 7-34).

In summary, it has been possible to synthesize new complexing agents, metal complexes and metal complex salts that open up new possibilities in diagnostic and therapeutic medicine.

The present invention also provides a novel class of ligands falling within Formula VI, as set forth below:

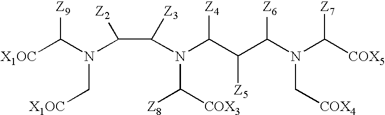

or a base or acid addition salt, hydrate, ester, solvate, prodrug, metabolite, stereoisomer, or mixture thereof, wherein at least one of Z₂ Z₃, Z₄, Z₅, Z₆, Z₇, Z₈ or Z₉ is a functionality suitable for coupling with a biocompatible macromolecule and the remaining Z₂ Z₃, Z₄, Z₅, Z₆, Z₇, Z₈ or Z₉ are non-coordinating substituents, X₁, X₂, X₃, X₄ and X₅ are independently OH or NR₂ wherein each R is a non-coordinating substituent, and at least two of X₁, X₂, X₃, X₄ and X₅ are OH.

Non-coordinating substituents include those that do not coordinate to a metal, such as alkyl groups and hydrogen atoms.

In some embodiments, the invention includes compounds of Formula IX or Formula VII:

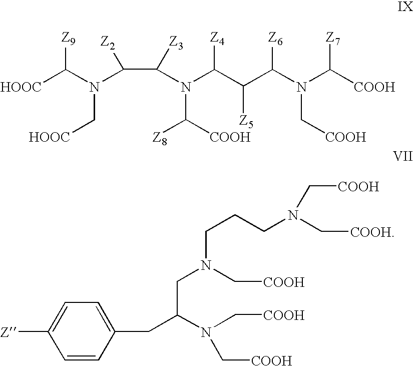

Preferably, the base or acid addition salt is a pharmaceutically acceptable salt. Salts encompassed within the term “pharmaceutically acceptable salt” are non-toxic salts of the compounds of this invention which are generally prepared by reacting an acidic complex with physiologically biocompatible cations of organic and/or inorganic bases or amino acids to produce “pharmaceutically-acceptable acid addition salts” of the compounds described herein. These compounds retain the biological effectiveness and properties of the free complexes. For example, the lithium ion, the potassium ion and especially the sodium ion are suitable inorganic cations. Suitable cations of organic bases include, among others, those of primary, secondary or tertiary amines, for example, ethanolamine, diethanolamine, morpholine, glucamine, N,N-dimethylglucamine or especially N-methylglucamine. Lysines, arginines or ornithines are suitable cations of amino acids, as generally are those of other basic naturally occurring such acids.

The metal atoms or cations, M, which are suitable for use in the complexes of the invention as MRI contrast agents are paramagnetic metals having atomic numbers 21-29, 42-44 and 57-71. The complexes for use as MRI contrast agents are those wherein the preferred metal is Eu, Gd, Dy, Ho, Cr, Mn or Fe, more preferably Mn(II) or Fe(III), and most preferably Gd(III).

The metal atoms or cations which are suitable for use in the complexes of the invention as X-ray or ultrasound contrast agents are heavy metals having atomic numbers 2032, 4244,49 and 57-83. The complexes for use as X-ray or ultrasound contrast agents are those wherein the preferred metal is a non-radioactive metal having atomic numbers 42-44, 49 and 57-83, most preferably Gd, Dy or Yb.

The metal atoms or cations of the complexes of the invention which are suitable for use in scintigraphic and radiotherapy are radioactive metals of any conventional complexable radioactive metal isotope, preferably those having atomic numbers 20-32, 42-44, 49 and 57-83. In scintigraphy, the most preferred metals are ^{99m}Tc or ¹¹¹In. In radiotherapy, the most preferred metals are ¹⁵³Sm, ⁶⁷Cu or ⁹⁰Y.

The metal atom or cations which are suitable for use as luminescence enhancers include, e.g., Eu and Tb.

Interaction of the H₅L ligand with Ln³⁺ ions (Ln=Lanthanides, e.g., Lanthanum (La), Europium (Eu), Lutetium (Lu), Gadolinium (Gd), and Terbium (Tb)) in dilute aqueous solution creates complexes with metal:ligand stoichiometry of 1:1. The 1:1 lanthanide complexes of the present invention display high thermodynamic stability under physiological conditions.

The compounds of the invention, including e.g., those of Formula VII, Formula IX and Formula VI, can be used to enhance images produced by method of magnetic resonance imaging. In one embodiment, a contrast medium made from a physiologically compatible complex of the invention and a nontoxic pharmaceutically acceptable carrier, adjuvant or vehicle is administered to a human or non-human animal (subject); and a magnetic resonance image is generated of at least a part of the subject. The compounds can similarly be used to enhance images produced by X-ray, ultrasound or scintigraphic imaging of a subject.

The methods of diagnostic analysis of the present invention involve administering the compounds of the invention to a human or non-human animal subject or host, in an amount sufficient to effect the desired contrast (or shift) and then subjecting the host to diagnostic analysis. Preferably diagnostic analysis is MRI analysis. Further, the complexes of the present invention are useful in diagnostic analysis by X-ray image analysis, ultrasonic analysis or scintigraphic analysis. While described primarily as contrast enhancing agents, the complexes of the invention can act as MRI shift reagents and such use is contemplated by the methods herein.

The complexes of the invention used as contrast enhancing agents are administered in an amount sufficient to effect the desired contrast. For MRI, this amount is an MRI signal effecting amount of the complex, i.e. any amount of said complex that will alter the spin-lattice (T1) or spin-spin or spin-echo (T2) relaxation times of an MRI signal. For a shift reagent, a sufficient amount of said complex will selectively shift the spectral position of a resonance nucleus relative to other similar nuclei. This alteration is effected in a manner in order to enhance the signals received from the subject under analysis either by reducing the aforementioned relaxation times or by increasing them with respect to an area of the host or the host per se which has had the complex administered to it. In another embodiment, the MRI signal effecting amount of the complex is that amount which in addition to changing the relaxation times of the MRI signals in the host, will also change such relaxation times sufficiently so that sharper lines of definition or higher contrast is obtained between those parts of the host that have and have not been administered the complex.

A detailed discussion of theoretical considerations for selecting the appropriate parameters for MRI diagnostic analysis is provided in U.S. Pat. No. 4,749,560, incorporated herein by reference. X-ray image analysis, ultrasonic diagnosis, scintigraphic image analysis and radiotherapy utilizing the complexes of the invention are all conducted in accordance with well-established techniques known to those of ordinary skill in the art.

The complexes of the invention may be administered to a host as a pharmaceutical composition in a contrast-enhancing amount. The pharmaceutical compositions contain a contrast-enhancing dosage of the contrast agents according to the invention together with a nontoxic pharmaceutically acceptable carrier, adjuvant or vehicle. The compositions can be administered by well-known routes including oral, intravenous, intramuscular, intranasal, intradermal, subcutaneous, parenteral, enteral and the like. Depending on the route of administration, the pharmaceutical composition may require protective coatings.

The pharmaceutical forms suitable for injectable use includes sterile solutions, suspensions, emulsions syrups or dispersions in oily or aqueous media and sterile powders for the extemporaneous preparation of sterile injectable solutions or dispersions. In all cases the ultimate solution form must be sterile and fluid. Typical carriers include a solvent or dispersion medium containing, for example, water, buffered aqueous solutions (i.e. biocompatable buffers), ethanol, polyol (glycerol, propylene glycol, polyethylene glycol, and the like), suitable mixtures thereof, surfactants or vegetable oils. Sterilization can be accomplished by any art recognized technique, including but not limited to, addition of antibacterial or antifungal agents, for example, paraben, chlorobutanol, phenol, sorbic acid, thimerosal, and the like. Further, isotonic agents, such as sugars or sodium chloride may be incorporated in the subject compositions.

Production of sterile injectable solutions containing the subject contrast agent is accomplished by incorporating these agents in the required amount in the appropriate solvent with various ingredients enumerated above, as required, followed by sterilization, preferably filter sterilization. To obtain a sterile powder, the above solutions are vacuum-dried or freeze-dried as necessary.

Solid dosage forms for oral administration may include capsules, tablets, pills, powders, granules and gels. In such solid dosage forms, the active compound may be admixed with at least one inert diluent such as sucrose, lactose or starch. Such dosage forms may also comprise, as in normal practice, additional substances other than inert diluent, e.g. lubricating agents such as magnesium stearate. In the case of capsules, tablets and pills, the dosage forms may also comprise buffering agents. Tablets and pills can additionally be prepared with enteric coatings. Liquid dosage forms for oral administration may include pharmaceutically acceptable emulsions, solutions, suspensions, syrups, and elixirs containing inert diluent commonly used in the art, such as water. Such compositions may also comprise adjuvants, such as wetting agents, emulsifying and suspending agents, and sweetening, flavoring, and perfuming agents.

The contrast agents of the inventions are thus compounded for convenient and effective administration in pharmaceutically effective amounts with a suitable pharmaceutically acceptable carrier, adjuvant or vehicle in a dosage which effects contrast enhancement. These amounts are preferably about 1 μmol to 1 mol of the contrast agent per liter and/or administered in doses of about 0.0001 to 5 mmol/kg body weight. Preferred compositions provide effective-dosages of contrast agents in the range of about 0.001-5 mmol/kg for MRI diagnostics, preferably about 0.00% 0.5 mmol/kg; in the range of about 0.1-5 mmol/kg for X-ray diagnostics; and in the range of about 0.1-5 mmol/kg for ultrasound diagnostics. For scintigraphic diagnostics, the dose of the contrast agent should generally be lower than for MRI diagnostics. For radiotherapy, conventional doses known to those of ordinary skill in the art can be used.

As used herein, a pharmaceutically acceptable carrier, adjuvant or vehicle includes any and all solvents, dispersion media, coatings, antibacterial and antifungal agents, isotonic agents, and the like. The use of such media and agents are well known in the art.

In some embodiments, ligand of Formula VII is coupled to a biological molecule prior to formation of the metal-ligand complex. In other embodiments, a ligand of Formula VII is coupled to a biological molecule after formation of the metal-ligand complex has been accomplished. These conjugates are particularly useful as image enhancing agents. Useful biological molecules are those known to concentrate in a particular organ or the part of an organ to be examined. These biomolecules include, for example, hormones (e.g., insulin), prostaglandins, steroid hormones, amino sugars, peptides, proteins, lipids, etc. Conjugates with albumins (e.g., human serum albumin) or antibodies, (e.g., monoclonal antibodies specific for tumor associated antigens or proteins such as myosin, etc.) are especially notable. The diagnostic agents formed therefrom are suitable, for example, for use in tumor and infarct diagnosis. Conjugates with liposomes, inclusion of salts of the contrast agent in liposomes, (e.g. unilamellar or multilamellar phosphatidylcholine-cholesterol vesicles) are suitable for liver imaging. The use of these complexes will allow tissue- or organ-specific diagnostic analysis of a subject. For example, the contrast enhancing agents can exhibit organ and tissue specificity, e.g. biodifferental distribution, such as in myocardial tissue when the complexes of the invention are lipophilic in nature.

The advantages of the new system described herein include applications in various fields of medicine, including angyology and in vivo temperature mapping. Furthermore, control of the aggregation of the spherical particles by changing the mass, size, shape and number of nanoparticles in solution may lead to further improvement of properties of the supramolecular aggregates, for instance the ability to reversibly control their relaxivity. Lin, et al., Nature, 1989, 339, 360. Thus, the molecules of the present invention are useful as both contrast agents for MRI and luminescent stains for medical mapping applications.

The details of one or more embodiments of the invention have been set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The following Examples are presented in order to more fully illustrate the preferred embodiments of the invention. These Examples do not limit the scope of the invention, as defined by the appended claims.

### EXAMPLE 1

a) 2-tert-Butoxycarbonylamino-3-(4-nitro-phenyl)-propionic acid methyl ester

C₁₅H₂₀N₂O₆ (M=324.34)

A suspension that consists of 50 g (161.0 mmol) of Boc-NO₂-Phe (Bachem), 40.5 g (483 mmol) of sodium bicarbonate and 25.0 g (177 mmol) of methyl iodide in 600 ml of dimethylformamide was stirred for four days at room temperature. The suspension was suctioned off, and the solid was washed with dichloromethane. The filtrate was concentrated by evaporation in a rotary evaporator. The residue was taken up in water and extracted four times with ethyl acetate (150 ml each). The combined, organic phases were washed with 100 ml of 5% sodium thiosulfate solution, with 100 ml of 5% sodium bicarbonate solution, with 50 ml of saturated sodium chloride solution, with 100 ml of 10% citric acid and 50 ml of water. The organic phase was dried on sodium sulfate, filtered and concentrated by evaporation in a rotary evaporator. A raw yield of 46.5 g (143.5 mmol), which corresponds to 89.4%, was produced.

Cld.: C 55.55H 6.22 N 8.64 O 29.60

Fnd.: C 55.59H 6.23 N 8.62 O 29.63

b) [2-Hydroxy-1-(4-nitro-benzyl)-ethyl]-carbamic acid tert-butyl ester

C₁₄H₂₀N₂O₅ (M=296.32)

8.9 g (27.4 mmol) of 1a was dissolved in 70 ml of tetrahydrofuran. 2.0 g (51.2 mmol) of sodium borohydride was added. 13 ml of methanol was slowly added in drops. The reaction solution was stirred overnight. The reaction solution was mixed with 2.8 ml of acetic acid and evaporated to the dry state. The residue was taken up in water and extracted with ethyl acetate. The combined organic phases were washed twice with saturated sodium chloride solution. The organic phase was dried on sodium sulfate, filtered off and concentrated by evaporation. Residual water with toluene was removed by azeotropic distillation in a rotary evaporator. The desired crude product was produced with a yield of 79.0% (6.4 g; 21.6 mmol).

Cld.: C 56.75H 6.80 N 9.45 O 27.00

Fnd.: C 56.69H 6.75 N 9.47 O 27.07

c) Methanesulfonic acid 2-tert-butoxycarbonylamino-3-(4-nitro-phenyl)-propyl ester

C₁₅H₂₂N₂O₇S (M=374.41)

6.40 g (21.6 mmol) of 1b was dissolved in 5 ml of dichloromethane and 0.33 g (3.24 mmol) of triethylamine. The solution was cooled to −5° C. and slowly mixed with 0.27 g (2.38 mmol) of methanosulfonic acid chloride, which was diluted in some dichloromethane. The suspension was stirred for two hours and poured onto stirred ice water (about 50 ml). The phases were separated. The aqueous phase was extracted three times with dichloromethane (50 ml each). The combined organic phases were washed twice with dilute (5%) aqueous HCl solution, with water, with dilute (5%) sodium bicarbonate solution and with saturated sodium chloride solution. The organic phase was dried on sodium sulfate and evaporated to the dry state in a rotary evaporator. A yield of 98.9% of crude product was produced.

Cld.: C 48.12H 5.92 N 7.48 O 29.91 S 8.56

Fnd.: C 48.21H 5.98 N 7.43 O 29.90 S 8.57

d) [2-(3-Amino-pentylamino)-1-(4-nitro-benzyl)-ethyl]-carbamic acid tert-butyl ester

C₁₉H₃₂N₄O₄ (M=380.49)

10 g (26.71 mmol) of 1c was reacted with 27.85 g (267.1 mmol) of 1,3-diamino-pentane, 2.96 g (29.3 mmol) of triethylamine and 100 ml of tetrahydrofuran analogously to Example 3a. The desired product was produced with a yield of 50.6% (5.14 g; 13.51 mmol). In addition, it was possible to isolate 3.75 g (9.87 mmol) of [2-(3-amino-1-ethyl-propylamino)-1-(4-nitro-benzyl)-ethyl]-carbamic acid tert-butyl ester.

Cld.: C 59.98H 8.84 N 14.73 O 16.82

Fnd.: C 59.90H 8.86 N 14.75 O 16.77

e) N′-[2-Amino-3-(4-nitro-phenyl)-propyl]-pentane-1,3-diamine

C₁₄H₂₄N₄O₂ (M=280.37)

3.65 g (9.59 mmol) of 1 d was dissolved in 55 ml of dichloromethane and mixed with 16.24 g (142.46 mmol) of trifluoroacetic acid. The solution was stirred for 90 minutes at room temperature and concentrated by evaporation in a rotary evaporator. It was mixed twice with dichloromethane and concentrated by evaporation again in each case. The crude product was mixed with 100 ml of 5% ammonia solution. The product was freeze-dried. The operating step described most recently was repeated. The desired product was produced with 2.39 g (8.54 mmol, 89%).

Cld.: C 59.98H 8.63 N 19.98 O 11.41

Fnd.: C 59.90H 8.62 N 19.92 O 11.44

f) {[2-{[3-(Bis-tert-butoxycarbonylmethyl-amino)-pentyl]-tert-butoxycarbonylmethyl-amino}-1-(4-nitro-benzyl)-ethyl]-tert-butoxycarbonylmethyl-amino}-acetic acid tert-butyl ester

C₄₄H₇₄N₄O₁₂ (M=851.08)

31.06 g (224.72 mmol) of potassium carbonate and 27.56 g (141.3 mmol) of bromoacetic acid-tert-butyl ester were added to a solution of 5.27 g (18.8 mmol) of 1 e in 246 ml of acetonitrile-water mixture (5:1). The reaction suspension was heated to 70° C. and stirred for 24 hours. The suspension was concentrated by evaporation in a rotary evaporator, mixed with 350 ml of water and extracted three times with ethyl acetate. The organic phase was dried on sodium sulfate, filtered and evaporated to the dry state in a rotary evaporator. The crude product was purified by column chromatography (SiO₂, dichloromethane->dichloromethane:methanol 8:1). The desired product was produced with a yield of 71.0% (11.36 g, 13.35 mmol).

Cld.: C 62.10H 8.76 N 6.58 O 22.56

Fnd.: C 61.98H 8.75 N 6.57 O 22.59

g) {[2-{[3-(Bis-carboxymethyl-amino)-pentyl]-carboxymethyl-amino}-1-(4-nitro-benzyl)-ethyl]-carboxymethyl-amino}-acetic acid

C₂₄H₃₄N₄O₁₂ (M=570.55)

20.18 g (23.71 mmol) of if was introduced into anisole. It was cooled to 0° C. 23 ml of trifluoroacetic acid was added. It was stirred at room temperature for 24 hours. The reaction solution was concentrated by evaporation. The residue was taken up in water and extracted three times with diethyl ether. The aqueous phase was mixed with 100 ml of 5% ammonia solution and freeze-dried. The operating step described most recently was repeated. The desired product was produced with a yield of 91% (12.31 g; 21.58 mmol).

Cld.: C 50.52H 6.01 N 9.82 O 33.65

Fnd.: C 50.42H 6.00 N 9.79 O 33.69

### EXAMPLE 2

a) N³-[2-Amino-3-(4-nitro-phenyl)-propyl]-pentane-1,3-diamine

C₁₄H₂₄N₄O₂ (M=280.37)

2.92 g (7.67 mmol) of isolated by-products from 1d was dissolved in 40 ml of dichloromethane and mixed with 12.99 g (114.0 mmol) of trifluoroacetic acid. The solution was stirred for 90 minutes at room temperature and concentrated by evaporation in a rotary evaporator. It was mixed twice with dichloromethane and concentrated by evaporation again in each case. The crude product was mixed with 100 ml of 5% ammonia solution and freeze-dried. The operating step mentioned most recently was repeated. The desired product was produced with a yield of 87% (1.87 g, 6.67 mmol).

Cld.: C 59.98H 8.63 N 19.98 O 11.41

Fnd.: C 59.89H 8.60 N 19.93 O 11.39

b) {[2-{[3-(Bis-tert-butoxycarbonylmethyl-amino)-1-ethyl-propyl]-tert-butoxycarbonylmethyl-amino}-1-(4-nitro-benzyl)-ethyl]-tert-butoxycarbonylmethyl-amino}-acetic acid tert-butyl ester

C₄₄H₇₄N₄O₁₂ (m=851.08)

11.80 g (85.4 mmol) of potassium carbonate and 10.47 g (53.7 mmol) of bromoacetic acid-tert-butyl ester were added to a solution of 6.18 g (7.14 mmol) of 2a in 246 ml of acetonitrile-water mixture (5:1). The reaction suspension was heated to 70° C. and stirred for 24 hours. The suspension was concentrated by evaporation in a rotary evaporator, mixed with 350 ml of water and extracted three times with ethyl acetate. The organic phase was dried on sodium sulfate, filtered off and evaporated to the dry state in a rotary evaporator. The crude product was purified by column chromatography (SiO₂, dichloromethane->dichloromethane:methane 8:1). The desired product was produced with a yield of 73.0% (4.44 g, 5.21 mmol).

Cld.: C 62.10H 8.76 N 6.58 O 22.56

Fnd.: C 61.98H 8.77 N 6.60 O 22.55

c) {[2-{[3-Bis-carboxymethyl-amino}-1-ethyl-propyl]-carboxymethyl-amino}-1-(4-nitro-benzyl)-ethyl]-carboxymethyl-amino}-acetic acid

C₂₄H₃₄N₄O₁₂ (M=570.55)

1.35 g (1.58 mmol) of 2b was introduced into anisole. It was cooled to 0° C. 1.53 ml of trifluoroacetic acid was added. It was stirred at room temperature for 24 hours. The reaction solution was concentrated by evaporation. The residue was taken up in water and extracted three times with diethyl ether. The aqueous phase was concentrated by evaporation, mixed with 100 ml of 5% ammonia solution and freeze-dried. The operating step mentioned most recently was repeated. The desired product was produced with a yield of 88% (793 mg, 1.39 mmol).

Cld.: C 50.52H 6.01 N 9.82 O 33.65

Fnd.: C 50.43H 6.02 N 9.80 O 33.61

### EXAMPLE 3

a) N-[2-(3-Amino-2,2-dimethyl-propylamino)-1-(4-nitro-benzyl)-ethyl]-2,2-dimethyl-propionamide

C₁₉H₃₂N₄O₃ (M=364.49)

13.8 g (134 mmol) of 1,3-diamino-2,2-dimethylpropane was added to a solution of 5.0 g (13.4 mmol) of mesylate from Example 1c in 5 ml of tetrahydrofuran and 2.1 ml (14.7 mmol) of triethylamine. The solution was heated for four hours to 50° C. The solution was concentrated by evaporation in a rotary evaporator. The residue was taken up in water and extracted three times with ethyl acetate. The organic phase was dried on sodium sulfate, filtered off, and concentrated by evaporation in a rotary evaporator. The residue was purified by column chromatography (SiO₂, dichloromethane->dichloromethane:methanol 1:1->methanol:ammonia (10%) 10:1), yield 73.2% (3.58 g, 9.80 mmol).

Cld.: C 62.61H 8.85 N 15.37 O 13.17

Fnd.: C 62.57H 8.86 N 15.39 O 13.17

b) N′-(3-Amino-2,2-dimethyl-propyl)-3-(4-nitro-phenyl)-propane-1,2-diamine

C₁₄H₂₄N₄O₂ (M=280.37)

5.23 g (13.75 mmol) of 3a was dissolved in 78 ml of dichloromethane and then mixed with 23.3 g (204.26 mmol) of trifluoroacetic acid. The solution was stirred for 90 minutes and concentrated by evaporation. The residue was taken up in 100 ml of 5% ammonia solution and freeze-dried. The operating step mentioned most recently was repeated. 8.21 g of product (13.2 mmol; 95.9%) was produced.

Cld.: C 59.98H 8.63 N 19.98 O 11.41

Fnd.: C 59.91H 8.60 N 19.91 O 11.45

c) [(3-{[2-(Bis-tert-butoxycarbonylmethyl-amino)-3-(4-nitro-phenyl)-propyl]-tert-butoxycarbonylmethyl-amino}-2,2-dimethyl-propyl)-tert-butoxycarbonylmethyl-amino]-acetic acid tert-butyl ester

C₄₄H₂₄N₄O₂ (M=851.08)

8.02 g (12.9 mmol) of 3b was dissolved in 170 ml of an acetonitrile-water mixture (5:1) and mixed with 21.23 g (153.6 mmol) of potassium carbonate and 13.6 g (69.7 mmol) of bromoacetic acid-t-butyl ester. The reaction solution was heated at 70° C. for 24 hours. The suspension was concentrated by evaporation in a rotary evaporator. The residue was taken up in 300 ml of water and washed three times with ethyl acetate. The organic phase was dried with sodium sulfate, filtered off and evaporated to the dry state in a rotary evaporator. The residue was purified by column chromatography (SiO₂, hexane-ethyl acetate 1:1). 9.79 g (11.5 mmol; 89.32%) of the product that is mentioned in the title was produced.

Cld.: C 62.10 H 8.76 N 6.58 O 22.56

Fnd.: C 62.19H 8.74 N 6.57 O 22.59

d) [(3-[([2-(Bis-carboxymethyl-amino)-3-(4-nitro-phenyl)-propyl]-carboxymethyl-amino]-2,2-dimethyl-propyl)-carboxymethyl-amino]-acetic acid

C₂₄H₃₄N₄O₁₂ (M=570.55)

5.30 g (6.23 mmol) of 3c was introduced into 43 ml of anisole at −5° C. 6.15 ml (79.8 mmol) of trifluoroacetic acid was added. It was stirred overnight at room temperature. The reaction solution was concentrated by evaporation. The residue was taken up in water and extracted three times with diethyl ether. The aqueous phase was mixed with 100 ml of 5% ammonia solution and freeze-dried. 2.95 g (5.17 mmol) of the desired product was produced. This corresponds to a yield of 83%.

Cld.: C 50.52H 6.01 N 9.82 O 33.65

Fnd.: C 50.43H 5.99 N 9.85 O 33.63

### Example 4

a) 3-Hydroxy-2-methyl-propionamide

C₄H₉NO (M=103.12)

Analogously to: J. Amer. Chem. Soc.; 117; 9; (1995); 2479-2490. A solution that consists of 30.0 g (225 mmol) of β-hydroxy-isobutyric acid methyl ester and 750 ml of a 9M ammoniacal methanol solution was stirred in an airtight glass vessel for 7 days at 50° C. The solution was concentrated by evaporation in a vacuum. The residue was washed with cold diethyl ether (a total of 200 ml). A white solid of 15.77 g (153 mmol; 68%) remained.

Cld.: C 46.59H 8.80 N 13.58 O 31.03

Fnd.: C 46.63H 8.83 N 13.55 O 31.06

b) 3-Amino-2-methyl-propan-1-ol

C₄H₁₁NO (M=89.14)

14.7 g (140 mmol) of 4a was suspended in 50 ml of tetrahydrofuran and mixed at 0° C. with 400 ml (400 mmol) of 1 M borane-THF-complex solution. The solution was refluxed for 4 hours and mixed at 0° C. with 70 ml of concentrated HCl solution. The solution was concentrated by evaporation in a rotary evaporator. Dilute sodium hydroxide solution (140 g of sodium hydroxide in 200 ml of water) was added at 0° C. to the solution. It was extracted four times with 100 ml each of chloroform. The combined organic phases were dried with magnesium sulfate, filtered and concentrated by evaporation. The residue was distilled in a water jet vacuum (92° C.). The desired product was produced with 9.23 g (103.6 mmol; 74%).

Cld.: C 53.90H 12.44 N 15.71 O 17.95

Fnd.: C 53.80H 12.42 N 15.68 O 17.98

c) (3-Hydroxy-2-methyl-propyl)-carbamic acid tert-butyl ester

C₉H₁₉NO₃ (M=189.25)

63.0 g (333 mmol) of 4b was dissolved in 240 ml of tetrahydrofuran and cooled to 0° C. At this temperature, 72.6 g (329.5 mmol) of di-tert-butyldicarbonate ((Boc)₂O), dissolved in 95 ml of THF, was added in drops. It was heated to room temperature and stirred for one hour. The solution was concentrated by evaporation in a rotary evaporator. The residue was taken up in 400 ml of diethyl ether and washed with 100 ml of 0.01 N HCl, 100 ml of water and with 100 ml of sodium bicarbonate solution (5%). The organic phase was dried on sodium sulfate, filtered and concentrated by evaporation in a rotary evaporator.

Cld.: C 57.12H 10.12 N 7.40 O 25.36

Fnd.: C 57.20H 10.10 N 7.40 O 25.39

d) Methanesulfonic acid 3-tert-butoxycarbonylamino-2-methyl-propyl ester

C₁₀H₂₁NO₅S (M=267.34)

29.7 ml (214.1 mmol) of triethylamine was added to 27.0 g (142.7 mmol) of 4c in 155 ml of dichloromethane. The solution was cooled to −5° C. and mixed with 11.7 ml (149.8 mmol) of methanesulfonic acid chloride, which had been dissolved ahead of time in 155 ml of dichloromethane. The suspension was stirred for two hours and mixed with 400 ml of water. The phases were separated. The aqueous phase was extracted twice with 150 ml each of dichloromethane. The combined, organic phases were washed twice with 200 ml of 0.1N HCl solution, once with 200 ml of 5% sodium bicarbonate solution and once with 100 ml of water. The organic phase was dried on sodium sulfate, filtered off and concentrated by evaporation in a rotary evaporator. The residue was recrystallized in 0° C. hexane. 34.7 g (129.9 mmol) of the desired product was produced; this corresponds to a yield of 91%.

Cld.: C 44.93H 7.92 N 5.24 O 29.92 S 11.99

Fnd.: C 44.90H 7.89 N 5.24 O 29.90 S 12.01

e) (3-Azido-2-methyl-propyl)-carbamic acid tert-butyl ester

C₉H₁₈N₄O₂ (M=214.27)

71.6 g (268.6 mmol) of 4d was dissolved in 490 ml of DMSO and mixed with 21.0 g (322.3 mmol) of sodium azide. The reaction solution was stirred for 24 hours at 40-45° C. The mixture was cooled to 25° C. and mixed with 500 ml of water. The solution was extracted five times with 250 ml of dichloromethane. The combined organic phases were washed twice with 150 ml each of saturated sodium chloride solution. The organic phase was dried with sodium sulfate, filtered off and concentrated by evaporation in a rotary evaporator. The residue was purified by column chromatography (SiO₂, hexane->hexane-ethyl acetate 1:1). After purification, 43.7 g (204 mmol) of product, which corresponds to a yield of 76%, was present.

Cld.: C 50.45H 8.47 N 26.15 O 14.93

Fnd.: C 50.51H 8.26 N 26.12 O 14.95

f) (3-Amino-2-methyl-propyl)-carbamic acid tert-butyl ester

C₉H₂₀N₂O₂ (M=188.27)

30.8 g (143.8 mmol) of 4e was dissolved in 412 ml of ethyl acetate and mixed with 4.5 g of Pd/C (10%). The reaction solution was stirred at 25° C. under hydrogen atmosphere for 24 hours. The solution was filtered and concentrated by evaporation in a rotary evaporator. The residue was purified by column chromatography (SiO₂, dichloromethane->dichloromethane:methanol 1:1). Yield: 24.28 g (129.0 mmol, 89.7%).

Cld.: C 57.42H 10.71 N 14.88 O 17.00

Fnd.: C 57.45H 10.73 N 14.90 O 16.99

g) {3-[2-tert-Butoxycarbonylamino-3-(4-nitro-phenyl)-propionylamino]-2-methyl-propyl}-carbamic acid tert-butyl ester

C₂₃H₃₆N₄O₇ (M=480.56)

21.7 g (115 mmol) of 4f was dissolved in 600 ml of dichloromethane/water (1:1) and mixed with 36.0 g (115 mmol) of Boc-protected nitrophenylalanine and 17.6 g (115 mmol) of 1-hydroxybenzotriazole-H₂O(HOBT). The solution was cooled to about −5° C. 24.0 g (127 mmol) of 1-(dimethylaminopropyl)-3-ethylcarbodiimide (EDCI) was added and stirred for 7 hours and for another three days at 25° C. The phases were separated. The aqueous phase was extracted twice with dichloromethane. The combined, organic phases were washed twice with 150 ml each of saturated sodium bicarbonate solution and some water. The organic phase was dried with the sodium sulfate, filtered off and concentrated by evaporation in a rotary evaporator. The residue was pulverized in a mortar and washed with cold hexane. It was dried in an oil pump. Yield: 35.1 g (73.1 mmol, 63.6%).

Cld.: C 57.49H 7.55 N 11.66 O 23.30

Fnd.: C 57.46H 7.55 N 11.67 O 23.32

h) 2-Amino-N-{3-amino-2-methyl-propyl}-3-(4-nitro-phenyl)-propionamide C₁₃H₂₀N₄O₃ (M=280.33) 10.3 g (21.4 mmol) of 4 g was suspended in 120 ml of dry dichloromethane. 24.5 ml (318 mmol) of trifluoroacetic acid was added in drops and stirred for one hour. The mixture was concentrated by evaporation in a rotary evaporator, mixed with 100 ml of dichloromethane and concentrated by evaporation again. The residue was washed with diethyl ether and dried at 40° C. in an oil pump. 100 ml of ammonia solution (5%) was added. It was freeze-dried. The desired product was produced with 5.55 g (19.80 mmol; 92.5%).

Cld.: C 55.70H 7.19 N 19.99 O 17.12

Fnd.: C 55.39H 7.21 N 19.89 O 17.19

i) (3-Amino-2-methyl-propyl)-[2-amino-3-(4-nitro-phenyl)-propyl]-amine dihydrochloride

C₁₃H₂₂N₄O₂ (M=252.32)

9.25 g (18.2 mmol) of 4 h was dissolved in 130 ml of absolute THF. 128.5 ml (128.5 mmol) of borane-THF complex (1 molar) was added in drops at 0° C. within 30 minutes. The solution was heated to room temperature and refluxed for 5 hours. The solution was cooled to 0° C., mixed with 35 ml of methanol, stirred for two hours and concentrated by evaporation. The residue was dissolved in 200 ml of ethanol, cooled in an ice bath and mixed with hydrogen chloride gas. The mixture was concentrated by evaporation and taken up in diethyl ether. The solid was suctioned off, flushed with diethyl ether and dried in a vacuum. 5.49 g (14.6 mmol: 80.3%) of the desired product was produced.

Cld.: C 41.56H 6.71 N 14.91 O 8.52 Cl 28.31

Fnd.: C 41.59H 6.76 N 14.92 O 8.54 Cl 28.26

k) [(3-{[2-(Bis-tert-butoxycarbonylmethyl-amino)-3-(4-nitro-phenyl)-propyl]-tert-butoxycarbonylmethyl-amino) -2-methyl-propyl)-tert-butoxycarbonylmethyl-amino]-acetic acid tert-butyl ester

C₄₃H₇₂N₄O₁₂ (M=837.06)

10.37 g (27.6 mmol) of 4i was dissolved in 300 ml of acetonitrile-water (5:1) and mixed with 45.5 g (329.2 mmol) of potassium carbonate. 29.1 g (149.4 mmol) of bromoacetic acid-tert-butyl ester was added. The batch was stirred at 70° C. for 24 hours. The reaction solution was mixed with 500 ml of water and extracted three times with 150 ml each of ethyl acetate. The organic phase was dried with sodium sulfate, filtered and concentrated by evaporation in a rotary evaporator. The residue was, purified by column chromatography (SiO₂, dichloromethane->dichloromethane:methanol 8:1). The desired product was produced with a yield of 73.0% (16.87 g; 20.1 mmol).

Cld.: C 61.70H 8.67 N 6.69 O 22.94

Fnd.: C 61.67H 8.66 N 6.71 O 22.91

l) [(3-{[2-(Bis-carboxymethyl-amino)-3-(4-nitro-phenyl)-propyl]-carboxymethyl-amino}-2-methyl-propyl)-carboxymethyl-amino]-acetic acid

C₂₃H₃₂N₄O₁₂=(M=556.52)

38.76 g (46.3 mmol) of 4k was introduced into 318 ml of anisole and cooled to −5° C. 458 ml of trifluoroacetic acid was added. It was stirred for 3 hours at 0° C. It was heated to room temperature and stirred for 24 hours. The reaction solution was concentrated by evaporation in a rotary evaporator. The residue was taken up in 250 ml of water and extracted three times with diethyl ether. The aqueous phase was concentrated by evaporation. Methyl/ammonia (5%) were added and again concentrated by evaporation. Then, it was dissolved in water and freeze-dried. The desired product was produced with a yield of 98.3% (40.9 g; 45.5 mmol).

Cld.: C 49.64H 5.82 N 10.07 O 34.50

Fnd.: C 49.53H 5.81 N 10.01 O 34.53

### EXAMPLE 5

a) 2-Benzyl-malonic acid diethyl ester

C₁₄H₁₆O₄ (M=250.29)

The synthesis of the desired product is known in the literature (Synthesis, 12, 2000, 1749-1755).

Cld.: C 67.18H 7.25 O 25.57

Fnd.: C 67.15H 7.26 O 25.54

b) 2-Benzyl-malonic acid amide

C₁₀H₁₂N₂O₂ (M=192.22)

The synthesis of the desired product has been performed analogously to Example 15b.

Cld.: C 62.49H 6.29 O 16.65 N 14.57

Fnd.: C 62.59H 6.30 O 16.64 N. 14.59

c) 2-Benzyl-propane-1,3-diamine

C₁₀H₁₆N₂ (M=164.25)

The reduction to the desired product has been performed analogously to Example 15c.

Cld.: 73.17H 9.82 N 17.06

Fnd.: 73.09H 9.85 N 17.09

d) [(2-Benzyl-3-{[2-(bis-carboxymethyl-amino)-3-(4-nitro-phenyl)-propyl]-carboxymethyl-amino}-propyl)-carboxymethyl-amino]-acetic acid

C₂₉H₃₆N₄O₁₂ (M=632.62)

The synthesis of the desired product and its precursors have been performed analogously to Example 15c.

Cld.: C 55.06H 5.74 N 8.86 O 30.35

Fnd.: C 55.09H 5.72 N 8.84 O 30.32

### EXAMPLE 6

Gd Complex of the Compound According to Example 3

GdNa₂C₂₄H₂₉N₄O₁₂ (M=768.74)

142.6 mg (0.25 mmol) of 3d was suspended in 4 ml of distilled water, heated to 80° C. and brought into solution. It was mixed in portions with 45.3 mg (0.125 mmol) of Gd₂O₃. The suspension was heated to 80° C. and stirred for one hour. The solution was cooled to room temperature and set at pH=7 with sodium hydroxide solution (1 M). The water was removed by freeze-drying. The desired was produced with 192.2 mg (0.25 mmol, 99.8%).

Cld.: C 37.50H 3.80 N 7.29 O 24.97 Gd 13.87 Na 5.98

Fnd.: C 37.49H 3.77 N 7.31 O 24.99 Gd 13.89 Na 6.01

### EXAMPLE 7

({3-[(2-(Bis-carboxymethyl-amino)-3-{4-[3-(2,5-dioxo-2,5-dihydro-pyrrol-1-yl)-propionylamino]-phenyl}-propyl)-carboxymethyl-amino]-2,2-dim ethyl-propyl}-carboxymethyl-amino)-acetic acid

C₃₁H₄₁N₅O₁₃ (M=691.69)

270.3 mg (0.5 mmol) of aniline derivative 8 and 328 ml (4.54 mmol) of N-methylmorpholine were dissolved in 2.5 ml of dimethyl sulfoxide and mixed with 134.16 mg (0.61 mmol) of activated ester of maleimide, MPHS (Fluka). The reaction solution was heated, so that a homogeneous solution was produced. It was stirred for 40 minutes and concentrated by evaporation in a vacuum. The residue was purified by RP-HPLC. 180 mg (0.26 mmol; 52%) of the desired product was obtained.

Cld.: C 53.83H 5.97 N 10.13 O 30.07

Fnd.: C 53.74H 6.00 N 10.08 O 30.09

### EXAMPLE 8

[(3{[3-(4-Amino-phenyl)-2-(bis-carboxymethyl-amino)-propyl]-carboxymethyl-amino)-2,2-dimethyl-propyl}-carboxymethyl-amino]-acetic acid

C₂₄H₃₆N₄O₁₀ (M=540.57)

998 mg (1.75 mmol) of 3d and 0.6 g of palladium on carbon (10%) were dissolved in 30 ml of methanol-water (4:1) and hydrogenated under hydrogen atmosphere (normal pressure) at room temperature until the calculated amount of hydrogen (39.2 ml) had been taken up. It was filtered and rewashed with methanol. It was concentrated by evaporation in a rotary evaporator, suspended in toluene and concentrated by evaporation again. The desired product was produced with 746 mg (1.38 mmol; 78.7% yield).

Cld.: C 53.33H 6.71 N 10.36 O 29.60

Fnd.: C 53.41H 6.74 N 10.42 O 29.61

### EXAMPLE 9

[(3-([2-(Bis-carboxymethyl-amino)-3-(4-isothiocyanato-phenyl)-propyl]-carboxymethyl-amino}-2,2-dimethyl-propyl)-carboxymethyl-amino]-acetic acid

C₂H₃₄N₄O₁₀S (M=582.63)

811 mg (1.5 mmol) of product of Example 8 and 969 mg (9.14 mmol) of sodium carbonate were dissolved in 35 ml of distilled water and 70 ml of chloroform. 132.8 ml (1.74 mmol) of thiophosgene was added to this 2-phase system. The solution was stirred for 3 hours. The solution was concentrated by evaporation, taken up in 0.1 ml of dilute acetic acid (1%), and purified by means of RP-HPLC (25:74:1 acetonitrile/water/acetic acid). The desired product was produced with a yield of 64.2% (561 mg; 963 mmol).

Cld.: C 51.54H 5.88 N 9.62 O 27.46 S 5.50

Fnd.: C 51.59H 5.88 N 9.64 O 27.50 S 5.48

### EXAMPLE 10

{[3-({2-(Bis-carboxymethyl-amino)-3-[4-(2-bromo-acetylamino)-phenyl]-propyl}-carboxymethyl-amino)-2,2-dimethyl-propyl].carboxymethyl-amino}-acetic acid

C₂₆H₃₇BrN₄O₁₁ (M=661.50)

81.1 mg (0.15 mmol) of product of Example 8 was dissolved in 3 ml of ethanol, 3 ml of distilled water and 3 ml of saturated sodium bicarbonate and mixed with 0.56 g (2.18 mmol) of bromoacetic acid anhydride. By the addition of solid sodium bicarbonate, the pH was kept at 8.5. The reaction solution was stirred for one hour and concentrated by evaporation. The residue was filtered over wadding, flushed with ethanol, concentrated by evaporation and purified by means of RP-HPLC. 79.4 mg (0.12 mmol) of product was produced. This corresponds to a yield of 80%.

Cld.: C 47.21H 5.64 N 8.47 O 26.60 Br 12.08

Fnd.: C 47.11H 5.68 N 8.49 O 26.54 Br 12.00

### EXAMPLE 11

{[3-({2-(Bis-carboxymethyl-amino)-3-[4-(2-iodo-acetylamino)-phenyl]-propyl}-carboxymethyl-amino)-2,2-dimethyl-propyl]-carboxymethyl-amino}-acetic acid

C₂₆H₃₇IN₄O₁₁ (M=708.50)

81.1 mg (0.15 mmol) of product of Example 8 was dissolved in 3 ml of ethanol, 3 ml of distilled water and 3 ml of saturated sodium bicarbonate and mixed with 771 mg (2.18 mmol) of iodoacetic acid anhydride. By the addition of solid sodium bicarbonate, the pH was kept at 8.5. The reaction solution was stirred for one hour and concentrated by evaporation. The residue was filtered over wadding, flushed with ethanol, concentrated by evaporation and purified by means of RP-HPLC. A yield of 69% (73.3 mg; 0.104 mmol) of the desired product was produced.

Cld.: C 44.08H 5.26 N 7.91 O 24.84 I 17.91

Fnd.: C 44.04H 5.29 N 7.89 O 24.75 I 17.99

### EXAMPLE 12

[(3-{[2-(Bis-carboxymethyl-amino)-3-(4-thiosemicarbazido-phenyl)-propyl]-carboxymethyl-amino}-2,2-dimethyl-propyl)-carboxymethyl-amino]-acetic acid

C₂₅H₃₈N₆O₁₀S (M=614.67)

The desired product was obtained from the compound of Example 8, analogously to the instructions in Collect. Czech. Chem. Commun., 57, 3, (1992), 656-659.

Cld.: C 48.85; H 6.23; N 13.67; O 26.03; S 5.22

Fnd.: C 48.77; H 6.25; N 13.62; O 26.06; S 5.25

### EXAMPLE 13

[(3-{[3-(4-Acetylamino-phenyl)-2-(bis-carboxymethyl-amino)-propyl]-carboxymethyl-amino}-2,2-dimethyl-propyl)-carboxymethyl-amino]-acetic acid

C₂₆H₃₈N₄O₁₁ (M=582.60)

Analogously to J. Amer. Chem. Soc., 120; 12; 1998; 2768-2779:

81.1 mg (0.15 mmol) of product of Example 8 was dissolved in 4.5 ml of acetonitrile-H₂O (9:1), cooled to 0° C. and mixed with 38.3 mg (375 mmol) of acetic acid anhydride. The solution was stirred for 4 hours at room temperature. It was filtered and concentrated by evaporation in a vacuum.

Cld.: C 53.60H 6.57 N 9.62 O 30.21

Fnd.: C 53.49H 6.54 N 9.64 O 30.24

### EXAMPLE 14

a) 2,3-Dimethyl-succinonitrile

C₆H₈N₂ (M=108.14)

The synthesis of 2,3-dimethyl-succinonitrile was performed according to a procedure by Whiteley and Marianelli (Synthesis (1978), 392-394) from 8.7 g (149.8 mmol) of acetone, 16 ml (150.4 mmol) of ethyl cyanoacetate and 10 g (154 mmol) of potassium cyanide. The crude product was purified by distillation in a vacuum. Yield: 7.0 g (64.7 mmol; 61%).

Cld.: C 66.64H 7.46 N 25.90

Fnd.: C 66.60H 7.44 N 25.92

b) 2,3-Dimethyl-butane-1,4-diamine C₈H₁₅N₂ (M=116.21)

The synthesis of 2,3-dimethyl-butane-1,4-diamine was performed according to instructions from Alzencang et al. (J. Med. Chem. 38; 21; 1995; 43374341): 10.81 mg (100 mmol) of 2,3-dimethyl-succinonitrile was reacted with saturated diborane-THF solution. 8.02 g (69 mmol; 69%) of desired product was produced as a colorless liquid.

C 62.02H 13.88 N 24.11

C62.19H 13.90 N 24.12

c) [(4-{[2-(Bis-carboxymethyl-amino)-3-(4-nitro-phenyl)-propyl]-carboxymethyl-amino}-2,3-dimethyl-butyl}-carboxymethyl-amino]-acetic acid

C₂₅H₃₆N₄O₁₂ (M=584.58)

The synthesis of the desired product and the corresponding precursors was performed analogously to Example 3.

Cld.: C 51.37H 6.21 N 9.58 O 32.84

Fnd.: C 51.34H 6.23 N 9.61 O 32.80

### EXAMPLE 15

a) Cyclopentane-1,1-dicarboxylic acid diethyl ester

CH₁₈O₄ (M=214.26)

The synthesis of the substance was performed from 1,4-dibromobutane and malonic acid diester according to instructions from J. Amer. Chem. Soc., 109; 22; 1987; 6825-6836.

Cld.: C 61.66H 8.47 O 29.87

Fnd.: C 61.69H 8.46 O 29.82

b) Cyclopentane-1,1-dicarboxylic acid diamide C₇H₁₂N₂O₂ (M=156.18)

A solution of 21.4 g (100 mmol) of 15a and 500 ml of a 9 M ammoniacal methanol solution was stirred in an airtight glass vessel for 7 days at 50° C. The solution was concentrated by evaporation in a vacuum. The residue was washed with cold diethyl ether (a total of 200 ml). A white solid of 10.0 g (64 mmol; 64%) remained.

Cld.: C 53.83H 7.74 N 17.97 O 20.49

Fnd.: C 53.80H 7.71 N 18.00 O 20.52

c) C-(1-Aminomethyl-cyclopentyl)-methylamine

C₇H₁₆N₂ (M=128.22)

9.9 g (63.4 mmol) of 15b was suspended in 50 ml of tetrahydrofuran. 400 ml (400 mmol) of 1 M-borane-THF complex solution was added at 0° C. The solution was refluxed for 4 hours and mixed at 0° C. with 70 ml of concentrated HCl solution. The solution was concentrated by evaporation in a rotary evaporator. Dilute sodium hydroxide solution (140 g of sodium hydroxide in 200 ml of water) was added at 0° C. to the solution. It was extracted four times with 100 ml each of chloroform. The combined, organic phases were dried with magnesium sulfate, filtered and concentrated by evaporation. The residue was distilled in a vacuum. The desired product was produced with 5.85 g (45.64 mmol; 72%).

Cld.: C 65.57H 12.58 N 21.85

Fnd.: C 65.49H 12.59 N 21.87

d) {[1-({[2-(Bis-carboxymethyl-amino)-3-(4-nitro-phenyl)-propyl]-carboxymethyl-amino}-methyl)-cyclopentylmethyl]-carboxymethyl-amino}-acetic acid

C₂₆H₃₆N₄O₁₂ (M=596.59)

The synthesis of the desired product and the corresponding precursors was performed analogously to Example 3 with the diamine 15c.

Cld.: C 52.35H 6.08 N 9.39 O 32.18

Fnd.: C 52.43H 6.09 N 9.40 O 32.21

### EXAMPLE 16

trans-[(4-{[2-(Bis-carboxymethyl-amino)-3-(4-nitro-phenyl)-propyl]-carboxymethyl-amino)-cyclohexyl)-carboxymethyl-amino]-acetic acid

C₂₅H₃₄N₄O₁₂ (M=584.58)

The synthesis of the desired product and the corresponding precursors was performed analogously to Example 3 with trans-1,4-diaminocyclohexane.

Cld.: C 51.37H 6.21 N 9.58 O 32.84

Fnd.: C 51.28H 6.23 N 9.60 O 32.88

### EXAMPLE 17

a) cis-2-Aminomethyl-cyclohexylamine

C₆H₁₄N₂ (M=114.19)

The compound has been produced as described in the literature (Tetrahedron; 44; 5; 1988; 1465-1476).

Cld.: C 65.57H 12.58 N 21.85

Fnd.: C 65.51H 12.61 N 21.87

b) {[2-[2-(Bis-carboxymethyl-amino)-cyclohexylmethyl]-carboxymethyl-amino}-1-(4-nitro-benzyl)-ethyl]-carboxymethyl-amino}-acetic acid

C₂₆H₃₆N₄O₁₂ (M=596.59)

The synthesis of the desired product and the corresponding precursors was performed analogously to Example 3 with the diamine 17a.

Cld.: C 52.35H 6.08 N 9.39 O 32.18

Fnd.: C 52.30H 6.09 N 9.36 O 32.20

### EXAMPLE 18

Antibody Conjugate of {[3-({2-(Bis-carboxymethyl-amino)-3-[4-(2-bromo-acetylamino)-phenyl]-propyl}-carboxymethyl-amino)-2,2-dimethyl-propyl]-carboxymethyl-amino}-acetic acid

200 μg of an antibody with freely accessible thiol groups (e.g., HuM195 (cf. Michael R. McDevitt, J. Nuc. Med. 40, 1999, 1772; commercially available from Protein Design Labs Inc., Mountainview, Calif., US)—if the antibody does not have any freely accessible thiol groups, the latter can be produced by the use of 2-iminothiolane HCl (e.g., EP 0 607 222 B1)) was diluted in 1.2 ml of borate buffer (50 mmol, pH 8.5), mixed with 238 μg (240 nmol) of product of Example 10, dissolved in 50 μl of borate buffer (see above), and stirred for 3 hours at 37° C. It was purified on a NAP-5 column (Amersham Pharmacia Biotech AB, Sephadex G-25, Mobile Phase: PBS).

### EXAMPLE 19

Indium 111-Labeled Antibody Conjugate of {[3-({2-(Bis-carboxymethyl-amino)-3-[4-(2-bromo-acetylamino)-phenyl]-propyl}-carboxymethyl-amino)-2,2-dimethyl-propyl]-carboxymethyl-amino}-acetic acid

200 μg of an antibody with freely accessible thiol groups (e.g., HuM195 (cf. Michael R. McDevitt, J. Nuc. Med. 40, 1999, 1722; commercially available from Protein Design Labs Inc., Mountainview, Calif., USA)—if the antibody does not have any freely accessible thiol groups, the latter can be produced by the use of 2-iminiothiolane HCl (e.g., EP 0 607 222 B1)) was diluted in 1.2 ml of borate buffer (50 mmol, pH 8.5), mixed with 238 μg (240 nmol) of product of Example 10, dissolved in 50 p, of borate buffer (see above), and stirred for 3 hours at 37° C. The borate buffer solution was exchanged for an acetate buffer, by the sample solution being set at 0.1 M (pH 6.0) three times for 1 hour in the Slide-A-Lyzer 10000, Pierce MWCO (dialysis process) against 200 ml of NaOAc buffer in each case. Finally, it was set at 0.1 M (pH 6) overnight against 400 ml of NaOAc buffer. The solution was mixed with 80 μl (0.05 M HCl) of [¹¹¹In]InCl₃ (27.88 MBq) and stirred for 30 minutes at room temperature. It was purified on an NAP-5 column (Amersham Pharmacia Biotech AB, Sephadex G-25, Mobile Phase: PBS).

### EXAMPLE 20

Yttrium 90-Labeled Antibody Conjugate of {[3-({2-(Bis-carboxymethyl-amino)-3-[4-(2-bromo-acetylamino)-phenyl]-propyl}-carboxymethyl-amino)-2,2-dim ethyl-propyl]-carboxymethyl-amino}-acetic Acid

200 μg of an antibody with freely accessible thiol groups (e.g., HuM195 (cf. Michael R. McDevitt, J. Nuc. Med. 40, 1999, 1722; commercially available from Protein Design Labs Inc., Mountainview, Calif., USA)—if the antibody does not have any freely accessible thiol groups, the latter can be produced by the use of 2-iminothiolane HCl (e.g., EP 0 607 222 B1)) was diluted in 1.2 ml of borate buffer (50 mmol, pH 8.5), mixed with 238 μg (240 nmol) of product from Example 10, dissolved in 50 μl of borate buffer (see above), and stirred for 3 hours at 37° C. The borate-buffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Finally, it was set at 0.1 M (pH 6) overnight against 400 ml of NaOAc buffer. The solution was mixed with 50 MBq of [⁹⁰Y]YCl₃ and stirred for 30 minutes at room temperature. It was purified on an NAP-5 column (Amersham Pharmacia Biotech AB, Sephadex G-25, Mobile Phase: PBS).

### EXAMPLE 21

The thermodynamic stability constant of the Gd(III) complex of 3,6,10-tri(carboxymethly)-3,6,10-triazadodecanedioic acid (e.g., the ligand of Formula VII, without the benzyl-Z linker group), determined by potentiometry is logo=22.77 (Wang et al, Dalton, 1998, 41131). For a DTPA analogue complexes, it is well-known that groups attached to the DTPA backbone do not interfere in stability (e.g. EOB-DTPA and DTPA). By analogy, for the Gd(III)(EPTPA-bz-NO₂) complex (the ligand according to Formula VII, where Z is a nitro group) one can also expect similar stability constant as the log determined for Gd(III)(EPTPA). This high stability ensures a very low toxicity (comparable to that of GdDTPA).

The Gd(EPTPA-bz-NO₂) complex (Formula VII, Z is NO₂) has been investigated by ¹⁷O NMR, EPR and ¹H NMRD. According to the ¹⁷O chemical shifts, the complex has one inner sphere water molecule. This result is supported by UV-Vis measurements performed on the Eu(III) complex. The water exchange rate obtained is kₑₓ²⁹⁸=1.4×10⁸ s⁻¹, 40 times higher than that measured on Gd(DTPA).

In order to attain very high proton relaxivities for Gd(III) complexes, one has to increase the rotational correlation time of the molecule and optimise water exchange rate. Whereas increasing the rotational correlation time is relatively easy with the application of macromolecules, it is more difficult to fine-tune the water exchange rate without reducing the thermodynamic stability of the complex. Consequently, proton relaxivities of macromolecular agents are very often limited by slow water exchange.

The water exchange rate obtained for Gd(EPTPA-bz-NO₂) is in the optimal range to attain high proton relaxivities. This property, together with the high thermodynamic stability, is a unique feature among Gd(III) chelates known so far and it makes Gd(EPTPA-bz-NO₂) an ideal synthon to be attached to any rigid, slowly tumbling macromolecular agent. Coupling this chelate to a macromolecule with a rotational correlation time of 30 ns (that of serum albumin e.g.) should attain relaxivities of 50 mM-1 s⁻¹ at 60 MHz proton Larmor frequency, although the monomer itself does not have high relaxivities. The proton relaxivities are shown in Table 1.

TABLE 1
Proton relaxivities (mM⁻¹ s⁻¹) of Gd(EPTPA-bz-NO₂)
as a function of the proton Larmor frequency.

Relaxivity

| ν(MHz) | 24.8° C. | 37.3° C. |
|---|---|---|
| 9.998 | 5.56 | 4.42 |
| 5.999 | 6.33 | 5.21 |
| 3.598 | 7.23 | 5.67 |
| 2.159 | 7.41 | 5.80 |
| 0.778 | 8.29 | 6.19 |
| 0.778 | 8.39 | 6.36 |
| 0.467 | 8.21 | 6.49 |
| 0.280 | 8.55 | 6.57 |
| 0.168 | 8.47 | 6.55 |
| 0.101 | 8.34 | 6.90 |
| 0.061 | 8.69 | 6.44 |
| 0.036 | 8.63 | 6.56 |
| 0.022 | 8.54 | 6.47 |
| 12.001 | 5.47 | 4.18 |
| 14.002 | 5.15 | 4.20 |
| 16.000 | 4.98 | 4.26 |
| 18.002 | 4.91 | 4.12 |
| 20.001 | 4.73 | |

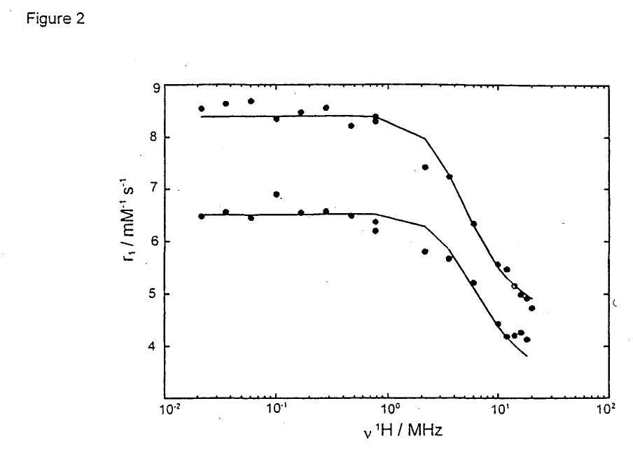

This information is presented graphically in FIG. 2, which shows the NMRD profiles of Gd(EPTPA-bz-NO₂) at 37° C. (bottom curve) and 25° C. (top curve).

The peak-to-peak line-widths of the EPR band of Gd(EPTPA-bz-NO₂) at 0.34 T measured as a function of temperature are shown in Table 2.

TABLE 2
Peak-to-peak line widths of the EPR band of Gd(EPTPA-bz-NO₂)
at 0.34 T measured as a function of the temperature.

| T (° C.) | ΔHₚₚ |
|---|---|
| 23.5 | 297.4 |
| 34.1 | 286 |
| 40.3 | 280.2 |
| 56.3 | 297.4 |
| 72.4 | 326 |
| 47.6 | 283.15 |
| 3 | 363.25 |

The variable temperature ¹⁷O NMR relaxation rates and chemical shifts are shown on Table 3.

TABLE 3
Variable temperature ¹⁷O NMR relaxation rates and chemical shifts.
GdEPTPA-bz-NO₂ (C = 55.79 mM, pH = 6.0, reference = HClO₄)

| | 1000/T/ | | | | | ν/Hz | | | |
|---|---|---|---|---|---|---|---|---|---|
| T/K | K⁻¹ | T₁/s(ref) | T₁/s | T₂/s(ref) | T₂/s | (ref.) | ν/Hz | Δw/Hz | Δwᵣ/Hz |
| 359.7 | 2.78 | 2.17E−02 | 1.89E−02 | 2.23E−02 | 9.67E−03 | −3707.2 | −3803.0 | −95.8 | −5.99E+05 |
| 338.7 | 2.95 | 1.58E−02 | 1.37E−02 | 1.77E−02 | 6.16E−03 | −3664.8 | −3764.2 | −99.4 | −6.22E+05 |
| 324.2 | 3.08 | 1.22E−02 | 1.04E−02 | 1.38E−02 | 4.25E−03 | −3630.3 | −3751.5 | −121.2 | −7.58E+05 |
| 306.7 | 3.26 | 8.69E−03 | 7.34E−03 | 9.21E−03 | 2.63E−03 | −3582.3 | −3702.7 | −120.4 | −7.53E+05 |
| 298.0 | 3.36 | 7.07E−03 | 5.99E−03 | 9.21E−03 | 2.63E−03 | −3582.2 | −3705.7 | −123.5 | −7.73E+05 |
| 293.5 | 3.40 | 6.10E−03 | 5.13E−03 | 7.14E−03 | 1.98E−03 | −3565.2 | −3690.4 | −125.25 | −7.84E+05 |
| 286.7 | 3.49 | 5.07E−03 | 4.28E−03 | 6.17E−03 | 1.67E−03 | −3560.6 | −3686.0 | −125.36 | −7.84E+05 |
| 274.5 | 3.64 | 3.56E−03 | 3.01E−03 | 5.17E−03 | 1.35E−03 | −3545.5 | −3678.6 | −133.1 | −8.33E+05 |

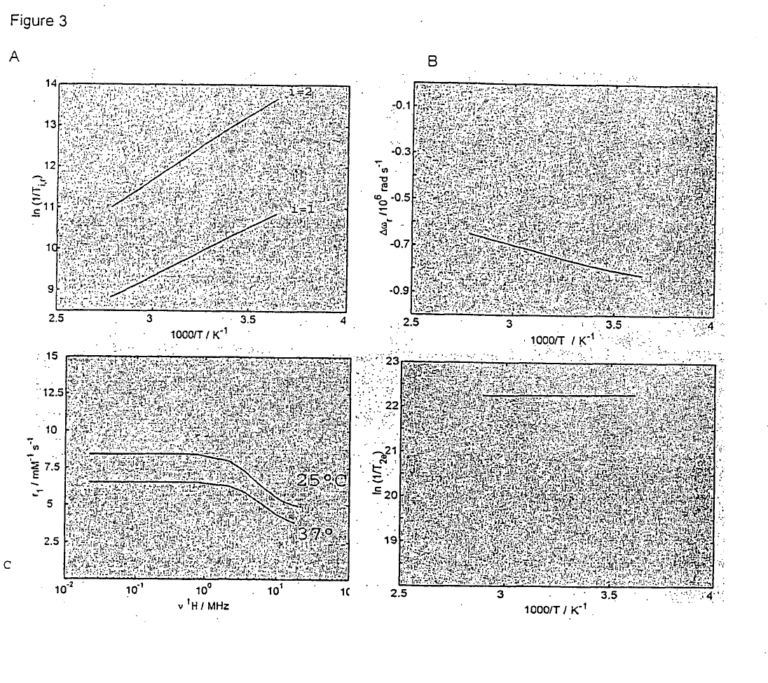

The ¹⁷O NMR, NMRD and EPR experimental data (points) and the fitted curves (lines) as obtained in a simultaneous analysis, according to the method of Powell et al., J. Am. Chem. Soc. 1996, 9333 are shown in FIG. 3:3A Top left: reduced transverse (i=2) and longitudinal (i=1) ¹⁷O relaxation rates (B=9.4 T), 3B: Top right: reduced ¹⁷O chemical shifts (B=9.4 T). 3C: Bottom left: NMRD profiles. 3D: Bottom right: transverse electron spin relaxation rates at 0.34 T, measured by EPR.

The parameters obtained in the simultaneous analysis of ¹⁷O NMR, EPR and NMRD data for Gd(EPTPA-bz-NO₂) and Gd(DTPA) are shown in Table 4.

TABLE 4

| Complexe de Gd(III) | DTPA | EPTPA-bz-NO₂ |
|---|---|---|
| ΔH^{‡}/kJ mol⁻¹ | 51.6 ± 1.4 | 23.7 ± 1.8 |
| ΔS^{‡}/J mol⁻¹K⁻¹ | 53.0 ± 4.7 | −9.2 ± 4.0 |
| kₑₓ²⁹⁸/10⁶ s⁻¹ | 3.3 ± 0.2 | 146 ± 23 |
| Eᵣ/kJ mol⁻¹ | 17.3 ± 0.8 | 19.9 ± 1.7 |
| τᵣ/ps | 58 ± 11 | 84.6 ± 6 |
| Eᵥ/kJ mol⁻¹ | 1.6 ± 1.8 | 1 0 |
| τ_{y}/ps | 25 ± 1 | 20 ± 1 |
| Δ²/10²⁰ s⁻¹ | 0.46 ± 0.02 | 0.37 ± 0.03 |
| A/ [symbol] /10⁶ rad s⁻¹ | −3.8 ± 0.2 | −3.5 ± 0.2 |

### EXAMPLE 22

The Ligand TRITA-bz-NO₂ (1) has been synthesized as described by Maecke and co-workers [G. Ruser, W. Ritter, H. R. Maecke, Bioconjugate Chem., 1990, 1, 345-349] using the commercially available dimethyl (4-nitrobenzyl)malonate [Aldrich] 2 and modifying the carboxymethylation reaction of the intermediate 12-(p-nitrobenzyl)-1,4,7,10-tetraazacyclotridecane 3 (see Scheme 1 below). The modified carboxymethylation was performed in the same way as described by Corson and Meares for a similar amine [D. T. Corson, C. F. Meares, Bioconjugate Chem., 2000, 11, 292-299] where the tert-butyl-ester groups were hydrolysed in 6M HCl at reflux. The final purification has been achieved by successive use of cation and anion exchange columns. The chelate contains a nitro-benzyl group which can function as a linker to couple the ligand to macromolecules according to well-documented procedures. This involves the transformation of the nitro-group to thiocyanate in molecule (1) which is then used for the coupling step.

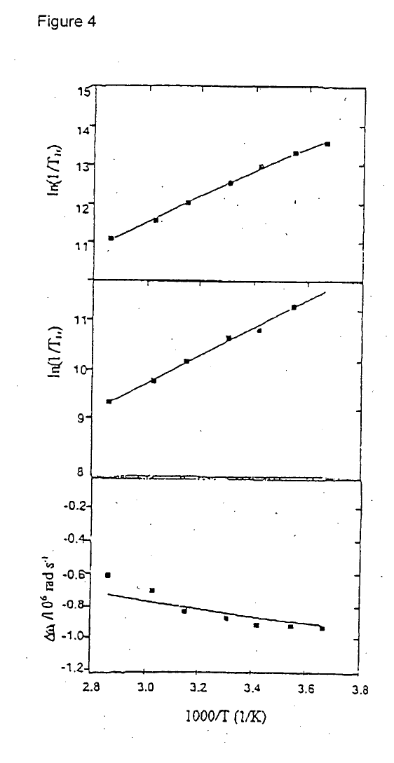

The rate of water exchange, Kₑₛ, has been measured on Gd(TRITA-bz-NO₂) by ¹⁷O NMR. The water exchange rate, as well as other parameters obtained from the analysis of the ¹⁷O NMR data are presented in Table 5. The experimental data as well as the fitted curves are given in FIG. 4. The numerical experimental data (reduced transverse and longitudinal ¹⁷O relaxation rates, reduced chemical shifts) measured on a Gd(TRITA-bz-NO₂) solution are shown in Table 6.

TABLE 5
Parameters obtained from the ¹⁷O NMR data on Gd(TRITA-bz-NO₂)
in comparison to the currently used MRI contrast agent Gd (DOTA)

| | Gd(DOTA)(H₂O)⁻ | Gd(TRITA-bz-NO₂)(H₂O)⁻ |
|---|---|---|
| kₑₓ²⁹⁸/10⁶ s⁻¹ | 4.1 | 180 ± 10 |
| ΔH^{x}/kJ mol⁻¹ | 49.8 | 26.8 ± 0.8 |
| ΔS*/Jmol⁻¹K⁻¹ | +10.5 | +3.0 ± 1.0 |
| A/ [symbol] /10⁶ rad s⁻¹ | −3.7 | −3.5 ± 0.2 |
| τR²⁹⁸/ps | 77 | 223 ± 20 |
| E_{R}/kJ mol⁻¹ | 16.1 | 23.3 ± 1.7 |

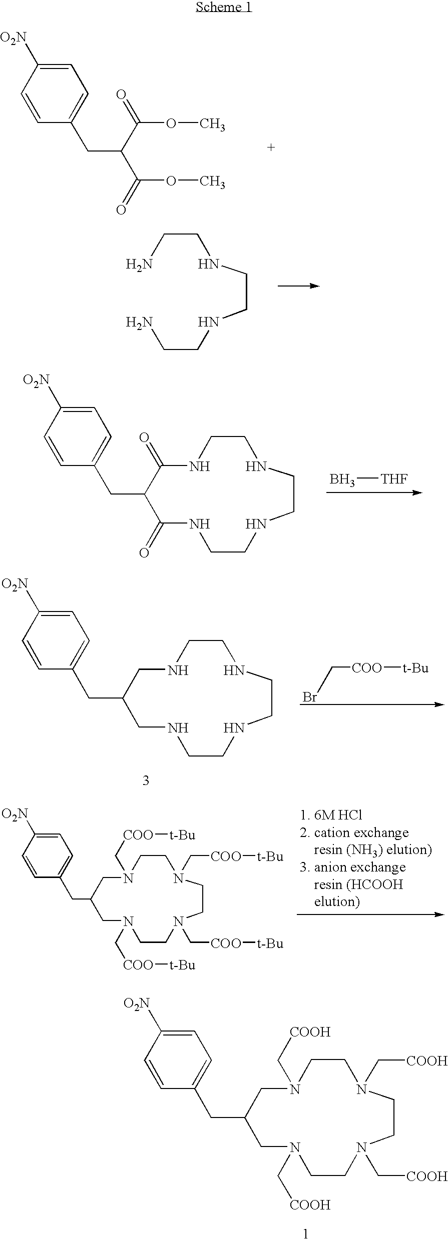

According to the ¹⁷O chemical shifts, the complex has 1 inner sphere water molecule. The water exchange rate obtained is Kₑₓ^{298-1.8×10}⁸ s⁻¹, over 40 times higher than that measured on Gd(DOTA).

The thermodynamic stability constant of the Gd(III) complex of TRITA (the same ligand without the linker group), determined by potentiometry is logβ=19.2 [Clarke and Martell, Inorg. Chim, Acta, 1991, 1990, 37-46]. For such type of poly(amino carboxylate) complexes it is well-known that gorups attached to the carbon backbone do not interfere in stability (e.g., EOB-DTPA and DTPA). By analogy, for the Gd(III)(TRITA-bz-NO₂) complex one can also expect similar stability constant as the logβ determined for Gd(III)(TRITA). This high stability ensures a very low toxicity.

In order to attain very high proton relaxivities for Gd(III) complexes, one has to increase the rotational correlation time of the molecules and optimise water exchange rate. Whereas increasing the rotational correlation time is relatively easy with the application of macromolecules, it is more difficult to fine-tune the water exchange rate without reducing the thermodynamic stabilty of the complex. Consequently, proton relaxivities of macromolecular agents are very often limited by slow ater exchange.

The water exchange rate obtained for Gd(III)(TRITA-bz-NO₂), this is a second example which shows that the elongation of the carbon backone by one CH₂ in DTPA- or DOTA-type ligands results in a considerable increase of the water exchange rate for the Gd(III) complex.

TABLE 6
Reduced transverse and longitudinal ¹⁷O relaxation rates and
chemical shifts measured on Gd(TRITA-bz-NO₂). B = 9.4 Tesla

| T/K | In(1/T₁ᵣ) | In(1/T₂ᵣ) | Δwᵣ/Hz |
|---|---|---|---|
| 302.25 | 10.44 | 12.54 | −8.72E+05 |
| 330.25 | 9.58 | 11.56 | −7.07E+05 |
| 349.45 | 9.14 | 11.06 | −6.13E+05 |
| 317.25 | 9.97 | 12.03 | −8.28E+05 |
| 273.05 | 11.42 | 13.55 | −9.34E+05 |
| 281.75 | 11.08 | 13.31 | −9.23E+05 |
| 292.45 | 10.60 | 12.96 | −9.15E+05 |

### EXAMPLE 23

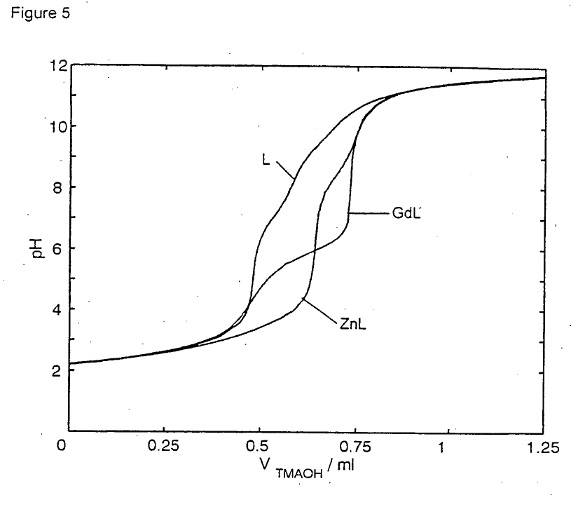

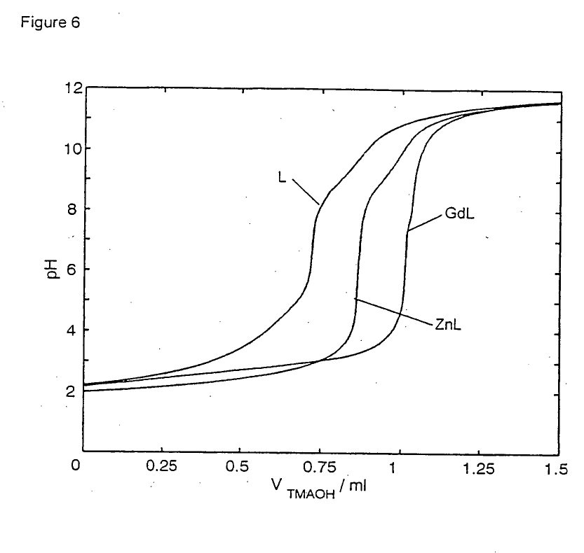

Protonation constants of the two ligands, DPTPA and EPTPA-Bz-NO₂, and stability constants of their complexes formed with Gd(III) and some endogenously available cations have been determined by pH-potentiometry. The selectivity of the ligand for Gd(III) over Zn(II) (log(K_{GdL}/K_{ZnL})) is especially important as it directly relates to the safety of the Gd(III) contrast agent. The results are summerized in Table 7. The titration curves of DPTPA and of the complexes MDPTPA (M=Gd and Zn; I=0.1M TMACI, t=25° C., CL=CM=2.06 mM) are shown in FIG. 5. The titration curves of EPTPABz-NO₂ and of the complexes M(EPTPA-Bz-NO₂) (M=Gd and Zn; I=0.1M TMACI, t=25° C., CL=CM=2.06 mM) are shown in FIG. 6.

TABLE 7
Protonation constants of the ligands and stability
constants of their complexes

| | EPTA-Bz-NO₂ | DPTPA | DTPA* |
|---|---|---|---|
| pKₐ₁ | 10.86 | 10.91 | 10.41 |
| pKₐ₂ | 8.94 | 9.29 | 8.37 |
| pKₐ₃ | 4.70 | 7.07 | 4.09 |
| pKₐ₄ | 3.25 | 2.76 | 2.51 |
| pKₐ₅ | 2.51 | 2.19 | 2.04 |
| logK_{GdL} | 19.31 | 13.00 | 22.50 |
| logK_{GdHL} | 3.35 | 6.30 | 1.80 |
| logK_{GdH2L} | 2.40 | 5.43 | |
| logK_{ZnL} | 16.01 | 15.60 | 18.3 |
| logK_{ZnHL} | 8.99 | 8.06 | 3.0 |
| logK_{ZnH2L} | 2.53 | 3.17 | |
| logK_{CaL} | 10.02 | 6.01 | 10.89 |
| logK_{CaHL} | 7.04 | 8.63 | 6.42 |
| logK_{CaH2L} | | 6.01 | |

*IUPAC Stability constants. Academic Software and K. J. Powell, 1999.

On elongation of the ligand skeleton, the thermodynamic stability of both the Gd(III) and Zn(II) complexes is decreasing. However, the EPTPA-Bz-NO₂ ligand ensures a stability for the lanthanide complex which is largely sufficient for biomedical use. DPTPA was prepared according to Scheme 2.

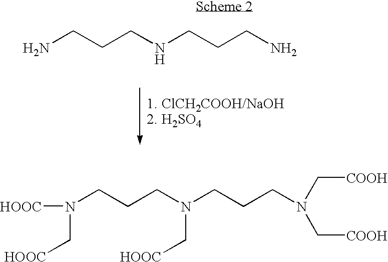

The foregoing description has been presented only for the purposes of illustration and is not intended to limit the invention to the precise form disclosed, but by the claims appended hereto.

1. Compounds of formula V 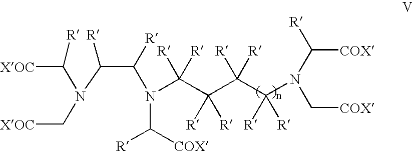
wherein n is 0 or 1; R′ are independently selected from the group consisting of a) functionalities suitable for coupling with a biocompatible macromolecule or biomolecule or b) non-coordinating substituents and at least one of R′ is a functionality suitable for coupling with a biocompatible macromolecule or biomolecule, whereby two of R′ in the propylene or butylene unit can be part of a 5- or 6-membered ring; and X′ are independently selected from the group consisting of OZ (wherein Z stands for a hydrogen atom or a metal ion equivalent) or NR₂ (wherein each R is a non-coordinating substituent); with the provisio that at least two of X′ are OZ;
or a salt, hydrate, ester, solvate, prodrug, metabolite, stereoisomer, or mixture thereof.

2. Compounds according to claim 1 of formula I 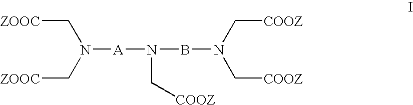
in which
Z stands for a hydrogen atom or a metal ion equivalent,
A stands for a radical of formula 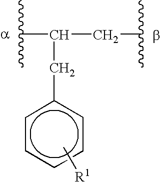
in which positions α and β that are characterized by 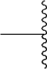
are bonded to any of the adjacent nitrogen atoms, R¹ is a nitro group or a group that can enter into a reaction with a biomolecule, and
B stands for a radical of formula 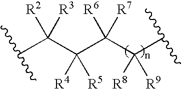
in which n is 0 or 1, and R², R³, R⁴, R⁵, R⁶, R⁷, R⁸ and R⁹, independently of one another, are selected from a hydrogen atom, a straight-chain or branched, saturated or unsaturated C₁ alkyl group, which optionally can be substituted with 1 or 2 hydroxy groups and/or can contain 1 or 2 oxygen atoms, and an aralkyl group, whose aryl radical optionally can be substituted with an alkyl or alkoxy group, whereby two of radicals R², R³, R⁴, R⁵, R⁶, R⁷, R⁸ and R⁹ can be part of a 5- or 6-membered ring, provided that at least one and at most four of radicals R², R³, R⁴, R⁵, R⁶, R⁷, R⁸, and R⁹ are not hydrogen atoms, as well as salts thereof.

3. Compounds according to claim 2, in which radical A is bonded via the α-position to the (ZOOC—CH₂)₂—N radical.

4. Compounds according to claim 2, in which R¹ is selected from the group that consists of nitro, amino, isocyanate, isothiocyanate, hydrazine, semicarbazide, thiosemicarbazide, chloroacetamide, bromoacetamide, iodoacetamide, acylamino, maleimide, maleimidacylamino, activated esters, mixed anhydrides, azide, hydroxide, sulfonyl chloride and carbodiimide.

5. Compounds according to claim 2, in which 1 or 2 of the radicals R², R³, R⁴, R⁵, R⁶, R⁷, R⁸ and R⁹ are selected from the group that consists of methyl, ethyl and benzyl, and the others of these radicals are hydrogen atoms.

6. Compounds claim 2, in which radicals R², R³, R⁴, R⁵, R⁶, R⁷, R⁸ and R⁹ are selected such that B is symmetrical.

7. Compounds according to claim 2, in which B is selected from the group that consists of —CH₂—CH₂—CH(CH₂—CH₃)—, —CH(CH₂—CH₃)—CH₂—CH₂—, —CH₂—C(CH₃)₂—CH₂—, —CH₂—CH(CH₃)—CH₂—, —CH₂—CH(CH₂-phenyl)-CH₂—, —CH₂—CH(CH₃)—CH(CH₃)—CH₂—,

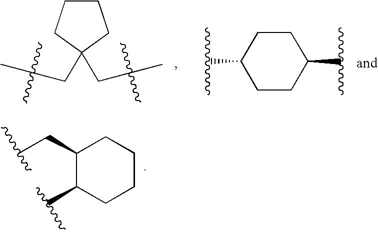

8. Compounds according to claim 1, in which n=0.

9. Compounds according to claim 1, in which at least two of radicals Z stand for a metal ion equivalent of a paramagnetic element of atomic numbers 21-29, 42, 44 and 58-70.

10. Compounds according to claim 1, in which at least two of radicals Z stand for a metal ion equivalent of a radioactive element of atomic numbers 26, 27, 29, 31, 32, 37-39, 43, 46, 47, 49, 61, 62, 64, 67, 70, 71, 75, 77, 82 and 83.

11. Conjugates of general formula II 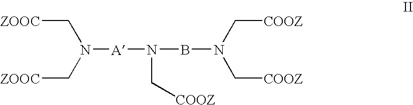
in which Z and B are defined as in claim 2, and A′ stands for a radical of formula 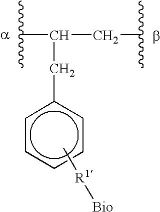
in which positions α and β that are characterized by 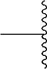
are bonded to any of the adjacent nitrogen atoms, and Bio stands for the radical of a biomolecule, which is bonded via radical R¹ of a reactive group to the phenylene ring, as well as salts thereof.

12. Conjugates according to claim 11, in which the biomolecule is selected from the group that consists of biopolymers, proteins, synthetically modified biopolymers, carbohydrates, antibodies, DNA and RNA fragments, β-amino acids, vector amines for transfer into the cell, biogenic amines, pharmaceutical agents, oncological preparations, synthetic polymers, which are directed to a biological target, steroids, prostaglandins, taxol and derivatives thereof, endothelins, alkaloids, folic acid and derivatives thereof, bioactive lipids, fats, fatty acid esters, synthetically modified mono-, di- and tri-glycerides, liposomes that are derivatized on the surface, micelles that consist of natural fatty acids or perfluoroalkyl compounds, porphyrins, texaphrines, expanded porphyrins, cytochromes, inhibitors, neuramidases, neuropeptides, immunomodulators, endoglycosidases, substrates that are attacked by the enzymes calmodolin kinase, casein-kinase II, glutathione-S-transferase, heparinase, matrix-metalloproteases, β-insulin-receptor-kinase, UDP-galactose, 4-epimerase, fucosidases, G-proteins, galactosidases, glycosidases, glycosyl transferases and xylosidases; antibiotics, vitamins and vitamin analogs, hormones, DNA-intercalators, nucleosides, nucleotides, lectins, vitamin B12, Lewis-X and related substances, psoralens, dienetriene antibiotics, carbacyclins, VEGF, somatostatin and derivatives thereof, biotin derivatives, antihormones, tumor-specific proteins and synthetic agents, dendrimers and cascade polymers, as well as derivatives thereof, polymers that accumulate in acidic or basic areas of the body, myoglobins, apomyoglobins, neurotransmitter peptides, tumor necrosis factors, peptides that accumulate in inflamed tissues, blood-pool reagents, anion and cation-transporter proteins, polyesters, polyamides and polyphosphates.

13. Conjugates according to claim 11, in which at least two of radicals Z stand for a metal ion equivalent of a paramagnetic element of atomic numbers 21-29, 42, 44 and 58-70.

14. Conjugates according to claim 11, in which at least two of radicals Z stand for a metal ion equivalent of a radioactive element of atomic numbers 26, 27, 29, 31, 32, 37-39, 43, 46, 47, 49, 61, 62, 64, 67, 70, 71, 75, 77, 82 and 83.

15. Use of a compound according to claim 1 for the production of a conjugate with a biomolecule.

16. Pharmaceutical agents that contains at least one physiologically compatible compound according to claim 9 or at least one physiologically compatible conjugate according to, optionally with the additives that are commonly used in galenicals.

17. Use of a compound according to claim 1 for the production of agents for NMR diagnosis.

18. Use of a compound according to claim 1 for the production of agents for radiodiagnosis or radiotherapy.

19. Kit for the production of radiopharmaceutical agents, comprising a compound according to claim 1, in which Z is hydrogen, and a compound of a radioactive element of atomic numbers 26, 27, 29, 31, 32, 37-39, 43, 46, 47, 49, 61, 62, 64, 67, 70, 71, 75, 77, 82 and 83.

20. Process for the production of a compound according to claim 2, in which a compound of formula III
H₂N-A-NH-B—NH₂ III
whereby A and B is reacted with a compound of formula IV
Nu-CH₂—COOZ′ IV
whereby Nu stands for a nucleofuge and Z′ stands for a hydrogen atom, a metal ion equivalent or a protective group for carboxyl, then the compound that is thus obtained is optionally reacted with a biomolecule, whereby the radical R¹, if it is nitro, first must be converted into a group that can enter into a reaction with a biomolecule and after that, and after the removal of optionally still present protective groups, and in a way that is known in the art, is reacted, if desired, with at least one metal oxide or metal salt and optionally then acidic hydrogen atoms that are still present in the complexes that are thus obtained are substituted completely or partially by cations of inorganic and/or organic bases, amino acids or amino acid amides.

21. A compound according to claim 1 of Formula VI 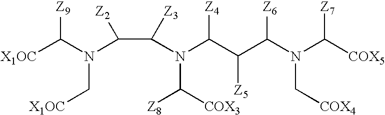
or a base or acid addition salt, hydrate, ester, solvate, prodrug, metabolite, stereoisomer, or mixture thereof, wherein at least one of Z₂ Z₃, Z₄, Z₅, Z₆, Z₇, Z₈, or Z₉ is a functionality suitable for coupling with a biocompatible macromolecule and each of the remaining Z₂ Z₃, Z₄, Z₅, Z₆, Z₇, Z₈, or Z₉ is a non-coordinating substituent;
X₁, X₂, X₃, X₄ and X₅ are independently OH or NR₂ wherein each R is a non-coordinating substituent;
with the proviso that at least two of X₁, X₂, X₃, X₄ and X₅ are OH.

22. The compound of claim 21, wherein each non coordinating substituent is, independently, a hydrogen.

23. The compound of claim 21, wherein each R is, independently, a hydrogen.

24. The compound of claim 21, wherein the functionality suitable for coupling with a biocompatible macromolecule is a isothiocyanato-benzyl.

25. The compound of claim 21, wherein each of X₁, X₂, X₃, X₄ and X₅ is OH.

26. A compound according to claim 1 of Formula VII: 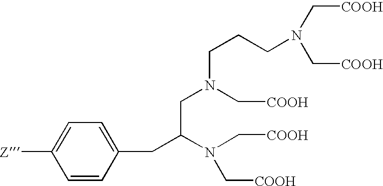
or a base or acid addition salt, hydrate, ester, solvate, prodrug, metabolite, stereoisomer, or mixture thereof, wherein Z″ is a hydrogen or a functionality suitable for coupling with a biocompatible macromolecule.

27. The compound of claim 26, wherein Z″ is selected from the group consisting of hydrogen, nitro and isothiocyanate.

28. A compound according to claim 1 of Formula VIII: 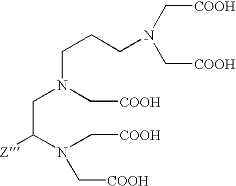
or a base or acid addition salt, hydrate, ester, solvate, prodrug, metabolite, stereoisomer, or mixture thereof, wherein Z′″ is a functionality suitable for coupling with a biological material or any biocompatible macromolecule.

29. The compound of claim 28, wherein the functionality suitable for coupling with a biological material is an isothiocyanate.

30. The compound of claim 28 where Z′″ is a benzyl group substituted by a functional group, capable of covalent or non-covalent binding to any biologically available or biocompatible material, or capable of self-aggregation.

31. The compound of claim 30 where one or two carboxylate groups are transformed to amide functions (any amide).

32. The compound of claim 21, wherein the base or acid addition salt is a pharmaceutically acceptable salt.

33. A complex between a compound of claim 21 and a metal.

34. The complex of claim 33, wherein the metal is selected from the group consisting of elements having atomic numbers 21-29, 42-44, and 57-83.

35. The complex of claim 33, wherein the metal is selected from the group consisting of di- and tri-positive metals having a coordination number from 2 to 9.

36. The complex of claim 35, wherein the metal is selected from the group consisting of tri-positive metals having a coordination number of 8 or 9.

37. The complex of claim 36, wherein the metal is selected from the group consisting of Lanthanum, Europium, Gadolinium, Terbium, and Lutetium.

38. The complex of claim 33, wherein the metal is Gadolinium (III).

39. The complex of claim 33 wherein Z″ or Z′″ is attached to a biocompatible macromolecule.

40. The complex of claim 39 wherein the biological molecule is a protein.

41. A method of magnetic resonance imaging a subject, the method comprising administering a complex of claim 33 and a non-toxic, pharmaceutically acceptable carrier, adjuvant, or other vehicle, and generating a magnetic resonance image of at least a part of said subject.

42. The method of claim 41, wherein the subject is a human patient.

43. A method of imaging a subject comprising the steps of (a) administering a contrast medium comprising a physiologically compatible complex of a ligand of VI as defined in claim 21: and an element selected from the group consisting of metals having atomic numbers 21-29, 42-44, and 57-83; and (b) obtaining an image of said patient.

44. The method of claim 43, wherein the imaging is magnetic resonance imaging.

45. The method of claim 44, wherein the element is selected from the group consisting of the Lanthanides.

46. The method of claim 45, wherein the element is Gadolinium.

47. The method of claim 46, wherein the subject is human.

48. The method of claim 43, wherein the imaging is X-ray.

49. The method of claim 48, wherein the element is Bismuth.

50. The method of claim 43, wherein the imaging is ultrasound imaging.

51. The method of claim 43, wherein the imaging is scintigraphic imaging.

52. A method of radioimmunotherapy of a human subject applying a complex of claim 33, wherein the biocompatible macromolecule is a protein and the metal is selected from the group consisting of elements having atomic numbers 26, 27, 29, 31, 32, 37-39, 43, 49, 62, 64, 70, 75, 77, 82 and 83.
